(19) United States
(12) Patent Application Publication
(10) Pub. No.: US 2024/0001041 A1
(43) Pub. Date: Jan. 4, 2024

(54) Drug Delivery Device

(71) Applicant: Sanofi, Paris (FR)

(72) Inventors: Uwe Dasbach, Frankfurt am Main (DE); Thomas Mark Kemp, Melbourn, Hertfordshire (GB); Timothy Denyer, Melbourn, Hertfordshire (GB); Robbie Wilson, Melbourn, Hertfordshire (GB); Chris Rosier, Melbourn, Hertfordshire (GB)

(21) Appl. No.: 18/038,368

(22) PCT Filed: Dec. 1, 2021
(86) PCT No.: PCT/EP2021/083826

§ 371 Date: May 23, 2023

(30) Foreign Application Priority Data
Dec. 2, 2020 (EP) 20315477.8

(51) Int. Cl. A61M 5/32; A61M 5/315

(52) U.S. Cl. CPC A61M 5/3243; A61M 5/31511

(57) ABSTRACT

A drug delivery device includes a housing element, a protection member, a movable member, wherein the drug delivery device has an initial state, in which (i) the protection member is in an extended position to cover the drug delivery element, (ii) the protection member is movable in a proximal direction from the extended position into a retracted position in order to expose the drug delivery element, and (iii) the protection member is coupled to the movable member in distal direction and proximal direction such that an axial movement of the protection member in both, the distal and proximal direction, results in an axial movement of the movable member in the same direction. The drug delivery device is configured such that, starting from the initial state, movement of the protection member in proximal direction initiates a rotation of the movable member relative to the protection member by a predetermined angle.

### CROSS REFERENCE TO RELATED APPLICATIONS

The present application is the national stage entry of International Patent Application No. PCT/EP2021/083826, filed on Dec. 1, 2021, and claims priority to Application No. EP 20315477.8, filed on Dec. 2, 2020, the disclosures of which are incorporated herein by reference.

### TECHNICAL FIELD

A drug delivery device is provided.

### BACKGROUND

Administering an injection is a process which presents a number of risks and challenges for users and healthcare professionals, both mental and physical. A drug delivery device may aim to make self-injection easier for patients. A conventional drug delivery device may provide the force for administering the injection by a spring, and trigger button or another mechanism may be used to activate the injection. Drug delivery devices may be single-use or reusable devices.

### SUMMARY

An improved drug delivery device is disclosed.

According to at least one embodiment, the drug delivery device comprises a housing element. The housing element may be hollow and/or elongated. The housing element may be a sleeve, e.g. a cylindrically-shaped sleeve. Particularly, the housing element may be a holder for an energy member such as a drive spring, i.e. an element in which an energy member can be stored. The energy member may be secured to the housing element, e.g. by fixing one end of the drive spring to the housing element.

According to at least one embodiment, the drug delivery device comprises a protection member arranged axially movable with respect to the housing element and configured to cover a drug delivery element. The protection member may be a needle shroud. For example, the protection member is telescopically coupled to the housing element. The protection member may be rotationally fixed with respect to the housing element.

The drug delivery element may be, e.g., a needle or a cannula or a catheter. The protection member may be configured such that an axial movement of the protection member in a proximal direction exposes the drug delivery element and an axial movement in a distal direction covers the drug delivery element.

Here and in the following, if not stated otherwise, a movement of a member or element or feature is to be understood as a movement with respect to the housing element.

According to at least one embodiment, the drug delivery device comprises a movable member arranged axially and rotationally movable with respect to the housing element. Preferably, the movable member is also arranged rotationally movable with respect to the protection member. The movable member may be hollow and/or elongated. The movable member may be a sleeve or collar. For example, the movable member is received in the housing element and is circumferentially surrounded, e.g. circumferentially completely surrounded, by the housing element.

According to at least one embodiment, the drug delivery device has an initial state, in the following also referred to as locked state or first locked state. The initial state is a state which the drug delivery device can occupy and/or into which the drug delivery device can be switched. The initial state may be the state in which the drug delivery device is delivered to a user.

According to at least one embodiment, in the initial state, the protection member is in an extended position to cover the drug delivery element. Thus, when the drug delivery device is provided with a drug delivery element, the protection member in the extended position may cover, e.g. completely cover, the drug delivery element. In the extended position, the protection member may project beyond the drug delivery element in distal direction and/or may circumferentially completely surround the drug delivery element.

According to at least one embodiment, in the initial state, the protection member is movable in a proximal direction from the extended position into a retracted position in order to expose the drug delivery element. In the retracted position, the drug delivery element may be exposed such that it can be pierced into a tissue of a body. In the retracted position, the drug delivery element may project beyond the protection member and/or a housing of the drug delivery device in distal direction.

According to at least one embodiment, in the initial state, the protection member is coupled to the movable member in distal direction and proximal direction such that an axial movement of the protection member in both, the distal direction and the proximal direction, results in an axial movement of the movable member in the same direction. In other words, in the initial state, the protection member and the movable member may be axially bidirectionally coupled. Preferably, the protection member and the movable member are directly coupled, i.e. without an intermediate element. Particularly, when the protection member and the movable member are coupled in an axial direction, a movement of the protection member in this axial direction over a certain distance results in a movement of the movable member in this axial direction by the same distance.

According to at least one embodiment, the drug delivery device is configured such that, starting from the initial state, a movement of the protection member in proximal direction initiates a rotation of the movable member relative to the protection member and/or relative to the housing element by a predetermined angle or certain angle, respectively. For example, the movable member rotates in a first rotational direction relative to the protection member and/or the housing element by the predetermined angle. The rotation of the movable member relative to the protection member by the predetermined angle may result in a release of the coupling of the protection member and the movable member in distal direction so that the protection member is movable back towards the extended position relative to the movable member. For example, after the rotation by the predetermined angle, the movable member does not follow the protection member when the protection member is moved in distal direction.

“Predetermined” means that the angle by which the movable member rotates is preferably not arbitrary but defined or controlled by the construction of the drug delivery device. For example, the predetermined angle is at least 2° or at least 5° or at least 10°. Additionally or alternatively, the predetermined angle may be at most 90° or at most 45° or at most 30°. The rotational axis of the movable member may define or coincide with a longitudinal axis of the drug delivery device.

The rotation of the movable member by the predetermined angle preferably happens at an intermediate position of the protection member lying in axial direction between the extended position and the retracted position, e.g. closer to the retracted position than to the extended position. The drug delivery device may be configured such that after the rotation by the predetermined angle, the movable member is disabled from a further rotation in the same rotational direction, e.g. by a rotation-lock interface coupling the movable member to the housing element.

The rotation of the movable member may be initiated by transforming part of the force used to move the protection member in proximal direction into a force acting on the movable member in a rotational direction, e.g. the first rotational direction. This may, e.g., be realized by a guiding feature, e.g. of the housing element, guiding the movable member in the rotational direction when being moved in proximal direction. The guiding feature may be a guide rail. Additionally or alternatively, the rotation of the movable member may be initiated by an energy member of the drug delivery device inducing a torque onto the movable member, the torque tending to rotate the movable member. The energy member may induce the torque onto the movable member already in the initial state.

Preferably, after the rotation of the movable member relative to the protection member by the predetermined angle, the movable member and the protection member are still coupled in proximal direction such that an axial movement of the protection member in proximal direction results in an axial movement of the movable member in proximal direction. In other words, after the rotation, the protection member and the movable member may be axially unidirectionally coupled, namely only coupled in proximal direction.

The drug delivery device may be configured such that, starting from the initial state, a movement of the protection member from the extended position into the retracted position and/or the rotation of the movable member by the predetermined angle activates the drug delivery device so that a drug stored in the drug delivery device is administered via the drug delivery element.

In at least one embodiment, the drug delivery device comprises a housing element and a protection member arranged axially movable with respect to the housing element and configured to cover a drug delivery element. The drug delivery device further comprises a movable member arranged axially and rotationally movable with respect to the housing element and arranged rotationally movable with respect to the protection member. The drug delivery device has an initial state in which the protection member is in an extended position to cover the drug delivery element, in which the protection member is movable in a proximal direction from the extended position into a retracted position in order to expose the drug delivery element and in which the protection member is coupled to the movable member in distal direction and in proximal direction such that an axial movement of the protection member in both distal and proximal direction results in an axial movement of the movable member in the same direction.

The drug delivery device is configured such that, starting from the initial state, a movement of the protection member in proximal direction initiates a rotation of the movable member relative to the protection member by a predetermined angle, whereby the coupling of the protection member and the movable member in distal direction is released so that the protection member is movable back towards the extended position relative to the movable member.

The decoupling of the movable member and the protection member by the rotation of the movable member may allow to move the protection member back towards or into the extended position in order to again cover the drug delivery element without the movable member blocking or preventing such a movement. The movable member may be used to initiate an activation of the drug delivery device, e.g. to release a locking mechanism and to start a drive mechanism for performing a drug delivery process.

The drug delivery device specified herein may be elongated and/or may comprise a longitudinal axis, i.e. a main extension axis. A direction parallel to the longitudinal axis is herein called an axial direction. By way of example, the drug delivery device may be cylindrically-shaped.

Furthermore, the drug delivery device may comprise a longitudinal end, which may be provided to face or to be pressed against a skin region of a human body. This end is herein called the distal end. A drug or medicament may be supplied via the distal end. The opposing longitudinal end is herein called the proximal end. The proximal end is, during usage, remote from the skin region. The axial direction pointing from the proximal end to the distal end is herein called distal direction. The axial direction pointing from the distal end to the proximal end is herein called proximal direction. A distal end of a member or element of the drug delivery device is herein understood to be the end of the member/element located most distally. Accordingly, the proximal end of a member or element is herein understood to be the end of the element/member located most proximally.

In other words, “distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is herein used to specify directions, ends or surfaces which are arranged or are to be arranged to face away from or point away from the dispensing end and/or from the distal end of the drug delivery device or components thereof. The distal end may be the end closest to the dispensing end and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be a needle end where a needle unit is or is to be mounted to the device, for example.

A direction perpendicular to the longitudinal axis and/or intersecting with the longitudinal axis is herein called radial direction. An inward radial direction is a radial direction pointing towards the longitudinal axis. An outward radial direction is a radial direction pointing away from the longitudinal axis.

The terms “angular direction”, “azimuthal direction” or “rotational direction” are herein used as synonyms. Such a direction is a direction perpendicular to the longitudinal axis and perpendicular to the radial direction.

An element or member or feature being rotationally, axially or radially fixed with respect to another element or member or feature means that a relative movement in rotational direction or axial direction or radial direction between the two elements/members/features is not possible or prevented.

The terms “protrusion” and “boss” are used as synonyms herein. The term “recess” may particularly stand for an indentation or a cut-out or opening or hole.

According to at least one embodiment, the movable member is rotated during movement of the protection member in proximal direction. For example, proximal movement of the protection member may be converted into rotational movement of the movable member via an interface between the movable member and the protection member.

According to at least one embodiment, the drug delivery device is an auto-injector.

According to at least one embodiment, the drug delivery device comprises a plunger rod being arranged axially movable with respect to the housing element. The plunger rod may be axially movable in only one or both axial directions. The plunger rod may be hollow or solid. The plunger rod may be cylindrically-shaped, e.g. hollow cylindrically-shaped. In case the plunger rod is hollow, further elements or members, e.g. other than an energy member for driving the plunger rod, may be received in the plunger rod.

According to at least one embodiment, the drug delivery device comprises an energy member configured to provide energy in order to induce an axial movement of the plunger rod in distal direction, preferably to drive the plunger rod. In other words, the energy member may be configured to provide energy to move the plunger rod in distal direction relative to the housing element. The energy member may be a drive spring, e.g. a torsion drive spring, particularly a spiral torsion spring or clock spring or power spring, or another component configured to induce a movement of the plunger rod, e.g. a gas cartridge or an electric motor. The drive spring may be formed of metal, e.g. steel. The longitudinal axis may run through the center of the drive spring.

The plunger rod and/or the energy member may be received in the housing element and may be circumferentially surrounded, e.g. circumferentially completely surrounded, by the housing element. The plunger rod may be received in the movable member and/or the energy member and may be circumferentially surrounded, e.g. circumferentially completely surrounded, by the movable member and/or the energy member. This may be the case at least in the initial state of the drug delivery device. The movable member may be located distally with respect to the energy member.

According to at least one embodiment, in the initial state, the plunger rod is coupled to the housing element via a lock interface, i.e. via at least one lock interface, which prevents an axial movement of the plunger rod induced by the energy member. The lock interface may be established by the movable member. In the initial state, the energy member may already induce a force onto the plunger rod. For example, the drive spring is already biased in the initial state.

In other words, in the initial state, a locking mechanism of the drug delivery device is locked and prevents a movement of the plunger rod in distal direction induced by the energy member. The movable member may be part of the locking mechanism.

According to at least one embodiment, the drug delivery device is configured to be switched from the initial state into a released state by moving the protection member from the extended position into the retracted position. During this movement, also the movable member is moved in proximal direction as explained above.

According to at least one embodiment, in the released state, the lock interface is released so that an axial movement of the plunger rod induced by the energy member is enabled. Particularly, the movement of the movable member in proximal direction may release the lock interface or the locking mechanism. The movable member may, in the following, also be referred to as activation member or activation collar.

According to at least one embodiment, in the released state, the plunger rod moves in distal direction due to the energy provided by the energy member.

According to at least one embodiment, the rotation of the movable member with respect to the protection member by the predetermined angle is induced by the energy member. Particularly, the energy member may induce a torque onto the movable member, e.g. already in the initial state, initiating the rotation when the movable member is moved in proximal direction, e.g. when the protection member is in the intermediate position.

According to at least one embodiment, the drug delivery device comprises a transfer member being arranged rotatably with respect to the housing element. The transfer member may also be arranged axially movable with respect to the housing element. The transfer member may be hollow and/or elongated. The transfer member may be a sleeve. For example, the transfer member is a rotating collar. The transfer member may be configured to be rotated in only one or both rotational directions. The rotational axis of the transfer member may define or may coincide with the longitudinal axis.

The plunger rod may be received in the transfer member so that the transfer member circumferentially surrounds, e.g. circumferentially completely surrounds, at least a portion of the plunger rod. The transfer member may be received in the housing element and/or in the energy member and/or in the movable member so that at least a portion of the transfer member is circumferentially surrounded, e.g. circumferentially completely surrounded, by the housing element and/or the energy member and/or the movable member.

The housing element and/or the protection member and/or the movable member and/or the plunger rod and/or the transfer member may comprise or consist of plastic. Each of them may be formed in one piece, e.g. be of unitary construction or integrally formed. Each of them may have a main extension direction parallel to the longitudinal axis. The longitudinal axis may run through the housing element and/or the protection member and/or the movable member and/or the plunger rod and/or the transfer member, e.g. through the center thereof.

According to at least one embodiment, the transfer member and the plunger rod are operatively coupled such that a rotation of the transfer member in the first rotational direction is converted into a movement of the plunger rod in distal direction.

According to at least one embodiment, in the released state, the energy member induces a torque onto the transfer member due to which the transfer member rotates in the first rotational direction and thereby forces the plunger rod to move axially in distal direction. In the initial state, the energy member may already induce a torque onto the transfer member. In the initial state, the transfer member may be prevented from a rotation by a rotation-lock interface. i.e. by at least one rotation-lock interface, coupling the transfer member to the housing element. The rotation-lock interface may be released due to the movement of the movable member and/or of the protection member in proximal direction.

According to at least one embodiment, the plunger rod and the transfer member are operatively coupled via a threaded interface. The threaded interface may be formed directly between the plunger rod and the transfer member. The threaded interface may transform a rotational movement of the transfer member into an axial movement of the plunger rod. For example, the plunger rod comprises a thread engaged with a thread of the transfer member. The thread of the plunger rod may be an external thread, the thread of the transfer member may be an internal thread, or vice versa. The transfer member may be axially secured to the housing element, e.g. via the energy member. For example, one end of the drive spring not fixed to the housing element is fixed to the transfer member. For example, one end of the drive spring not fixed to the housing element is fixed to the transfer member. For example, the transfer member is secured to the housing element such that a force necessary for moving the transfer member in one or both axial directions, particularly in the proximal direction, is greater than a force necessary to axially move the plunger rod.

According to at least one embodiment, the plunger rod is rotationally fixed to the housing element, e.g. via a splined interface. This means that the plunger rod does not rotate or is prevented from rotating during movement in axial direction. The splined interface may be formed directly between the plunger rod and the housing element. For example, the plunger rod has a splining element and the housing element has a splining element, e.g. complementarity to and/or mating with the splining element of the plunger rod. The splining elements of the plunger rod and the housing element may engage with each other, e.g. form-lockingly, thereby preventing the rotation of the plunger rod with respect to the housing element. One of the splining elements of the housing element and of the plunger rod may be a groove and the other one of the splining elements of the housing element and the plunger rod may be a protrusion. The protrusion may then engage or project into the groove thereby preventing rotation of the plunger rod. The groove may extend parallel to the longitudinal axis. For example, the groove is formed in the plunger rod and the protrusion is part of the housing element.

Preferably, the splined interface is in close proximity to the threaded interface, e.g. with a distance of at most 1 cm or at most 0.5 cm or at most 0.2 cm. This is beneficial since the torque on the plunger rod is resolved over a short distance reducing the stresses within the plunger rod.

According to at least one embodiment, in the released state, the transfer member rotates by an angle greater than or equal to any one of the following values: 60°, 80°, 120°, 180°, 270°, 360°. For example, in the released state, the transfer member rotates by at least n-times 360°, wherein n is an integer greater than or equal 1. For example, n is one of: 1, 2, 3, 4, 5, 6, 7, 8, 9, 10.

According to at least one embodiment, in the initial state, the movable member and the housing element are coupled via a first rotation-lock interface. The first rotation-lock interface may prevent a rotation of the movable member, e.g. in the first rotational direction, with respect to the housing element. The first rotation-lock interface may be formed directly between the movable member and the housing element.

According to at least one embodiment, in the initial state, the movable member and the transfer member are coupled via a second rotation-lock interface. The second rotation-lock interface may prevent a rotation of the transfer member, e.g. in the first rotational direction, with respect to the movable member. The second rotation-lock interface may be formed directly between the movable member and the transfer member.

When both, the first rotation-lock interface and the second rotation-lock interface are established, rotation of the transfer member, e.g. in the first rotational direction, with respect to the housing element is prevented. Thus, a movement of the plunger rod in distal direction driven by the energy member may be prevented.

According to at least one embodiment, the first rotation-lock interface and/or the second rotation lock-interface are released by moving the movable member in proximal direction. For example, when moving the movable member in proximal direction, first, the first rotation-lock interface is released and afterwards, e.g. after the movable member has been moved by a further distance in proximal direction, the second rotation-lock interface is released. For example, when the first rotation-lock interface is released, the movable member rotates by the predetermined angle, particularly induced by the energy member.

According to at least one embodiment, when moving the protection member together with the movable member in proximal direction, the protection member and the movable member are moved in proximal direction relative to the transfer member. For example, the transfer member is not moved axially during the movement of the protection member and the movable member in the proximal direction.

In the initial state, the energy member may induce a torque onto the transfer member and, due to the second rotation-lock interface, this torque is transferred to the movable member. The torque on the movable member may be absorbed by the coupling to the housing element via the first rotation-lock interface.

According to at least one embodiment, the movable member has a first rotation-lock feature configured to engage with a rotation lock-feature of the housing element. The engagement between the first rotation-lock feature of the movable member and the rotation-lock feature of the housing element prevents a rotation of the movable member, e.g. in the first rotational direction, with respect to the housing element. For example, in the initial state, the first rotation-lock feature of the movable member and the rotation-lock feature of the housing element are engaged. The engagement between the two rotation-lock features may establish the first rotation-lock interface.

According to at least one embodiment, the movable member has a second rotation-lock feature configured to engage with a rotation-lock feature of the transfer member. The engagement between the second rotation-lock feature of the movable member and the rotation-lock feature of the transfer member may prevent a rotation of the transfer member, e.g. in the first rotational direction, with respect to the movable member. For example, in the initial state, the second rotation-lock feature of the movable member and the rotation-lock feature of the transfer member are engaged. The engagement between the two rotation-lock features preferably establishes the second rotation-lock interface.

According to at least one embodiment, the drug delivery device is configured such that, starting from the initial state, a movement of the protection member in proximal direction first results in a disengagement of the first rotation-lock feature of the movable member and the rotation-lock feature of the housing element and later on results in a disengagement of the second rotation-lock feature of the movable member and the rotation-lock feature of the transfer member. The disengagement of the first rotation-lock feature of the movable member and the rotation-lock feature of the housing element may enable the rotation of the movable member by the predetermined angle. The disengagement of the second rotation-lock feature of the movable member and the rotation-lock feature of the transfer member may enable a rotation of the transfer member in the first rotational direction, e.g. by at least one of the above listed angles, particularly by at least 360°.

According to at least one embodiment, a slide feature is assigned to and arranged axially behind at least one of the first rotation-lock feature of the movable member and the rotation-lock feature of the housing element. For example, the slide feature is adjoining the assigned rotation-lock feature in axial direction. The slide feature may be axially and/or rotationally and/or radially fixed to the movable member or the housing element or may be part of the movable member or the housing element.

In case the slide feature is assigned to the movable member, the slide feature is preferably arranged behind the first rotation-lock feature of the movable member in distal direction. In case the slide feature is assigned to the housing element, the slide feature is preferably arranged behind the rotation-lock feature of the housing element in proximal direction.

According to at least one embodiment, the slide feature is arranged and configured such that, after disengagement of the first rotation-lock feature of the movable member and the rotation-lock feature of the housing element, the respective other rotation-lock feature abuts against and slides along the slide feature for a controlled rotation of the movable member relative to the protection member by the predetermined angle, e.g. in the first rotational direction. The slide feature may guide the rotation of the movable member. The slide feature may be a beveled surface against which the respective other rotation-lock feature abuts and along which it slides.

According to at least one embodiment, a further rotation-lock feature is arranged axially behind the slide feature. The further rotation-lock feature may be part of the movable member or of the housing element. If the further rotation-lock feature is part of the movable member, the further rotation-lock feature and the rotation-lock feature of the housing element may engage after the rotation of the movable member by the predetermined angle. This engagement may prevent a further rotation of the movable member, e.g. in the first rotational direction. Likewise, if the further rotation-lock feature is part of the housing element, the further rotation-lock feature and the first rotation-lock feature of the movable member may engage after the rotation of the movable member by the predetermined angle and this engagement may prevent a further rotation of the movable member, e.g. in the first rotational direction.

According to at least one embodiment, one of the first rotation-lock feature of the movable member and the rotation-lock feature of the housing element is a slit. The slit preferably has a main extension direction parallel to the longitudinal axis. Preferably, a width of the slit, measured in rotational direction, is constant over the entire axial extension of the slit. For example, the first rotation-lock feature of the movable member is a slit formed in the movable member.

According to at least one embodiment, the other one of the first rotation-lock feature of the movable member and the rotation-lock feature of the housing element is a protrusion, particularly a rib. The protrusion preferably projects in radial direction, e.g. in radial inward direction. A main extension direction of the rib is preferably parallel to the longitudinal axis. When engaged, the protrusion projects into the slit. For example, the rotation-lock feature of the housing element is a protrusion of the housing element.

According to at least one embodiment, the slide feature is a beveled surface tilted with respect to the longitudinal axis and/or with respect to the rotational direction. The beveled surface may run parallel to the radial direction. By way of example, an angle between the beveled surface and the longitudinal axis and/or the rotational direction is at least 5° and at most 85°.

For example, the movable member or the housing element comprises a recess wherein the slit is a first section of the recess. The recess may comprise a second section which adjoins the slit and in which the width of the recess increases starting from the slit in a direction away from the slit. The width is measured along the rotational direction. The slide feature may be a surface of the movable member or the housing element delimiting the recess in the second section in rotational direction.

According to at least one embodiment, the second rotation-lock feature of the movable member is a protrusion. The rotation-lock feature of the transfer member may also be a protrusion. For example, the second rotation-lock feature of the movable member is a protrusion projecting radially inwardly and the rotation-lock feature of the transfer member is a protrusion projecting radially outwardly. When engaged, the two protrusions may abut against each other in rotational direction so that a rotation of the transfer member, e.g. in the first rotational direction, with respect to the movable member is prevented.

According to at least one embodiment, the drug delivery device is configured such that, starting from the initial state, the movement of the protection member with the movable member coupled thereto from the extended position towards the retracted position results in an axial coupling between the movable member and the housing element. The axial coupling between the movable member and the housing element may be established before the protection member reaches the retracted position or at the moment the protection member reaches the retracted position. For example, the axial coupling happens after the rotation of the movable member by the predetermined angle. The axial coupling between the movable member and the housing element may be bidirectionally so that the movable member is coupled to the housing element in proximal direction and distal direction. The coupling is preferably such that the movable member cannot be moved relative to the housing element in proximal and/or distal direction anymore.

According to at least one embodiment, the protection member comprises a coupling feature. The coupling feature may be arranged in the region of the proximal end of the protection member. The coupling feature may be a flexible or resilient arm with a protrusion, e.g. the protrusion projecting in inward radial direction. The arm may be oriented axially with a main extension direction in axial direction. A distal end of the arm may be radially and/or axially and/or rotationally fixed to the rest of the protection member, e.g. a main body of the protection member. A proximal end of the arm may be displaceable in radial direction. The protrusion may be arranged closer to the proximal end than to the distal end of the arm.

According to at least one embodiment, the movable member comprises a first coupling feature. The first coupling feature may be a recess in the movable member. The first coupling feature may be arranged in the region of the distal end of the moveable member.

According to at least one embodiment, the coupling feature of the protection member and the first coupling feature of the movable member are configured to releasably engage in order to provide the releasable coupling between the movable member and the protection member. In the initial state, the two coupling features may be engaged such that the engagement provides the coupling in proximal direction and distal direction. After the rotation of the moveable member relative to the protection member by the predetermined angle, the two coupling features may still be engaged such that the engagement only provides a coupling in proximal direction but not in distal direction. After the rotation by the predetermined angle, a movement of the protection member in distal direction relative to the movable member may result in a complete disengagement of the two coupling features. For example, when engaged, the protrusion of the arm projects into the recess.

According to at least one embodiment, at least one of the coupling features of the protection member and the first coupling feature of the movable member comprises a first section and a second section arranged behind each other in rotational direction. The coupling feature with the two sections may be formed differently in the two sections.

According to at least one embodiment, when the first section of the coupling feature is engaged with the other coupling feature, the protection member and the movable member are coupled in distal direction and proximal direction. This means, a movement of the protection member in proximal direction or in distal direction results in a movement of the movable member in the same direction, respectively.

According to at least one embodiment, when the second section of the coupling feature is engaged with the other coupling feature, the protection member and the movable member are only coupled in proximal direction and/or decoupled in distal direction. This means, a movement of the protection member in proximal direction results in a movement of the movable member in proximal direction. On the other hand, a movement of the protection member in distal direction relative to the movable member is enabled or a movement of the protection member in distal direction does not result in a movement of the movable member in distal direction, respectively.

By way of example, the first coupling feature of the movable member comprises the two sections. For example, the first coupling feature of the movable member is an L-shaped recess in the movable member. In the first section, the recess is delimited by edges of the movable member in proximal direction and in distal direction. In the second section, the recess may be delimited by an edge of the movable member only in proximal direction. For example, in the second section, the recess is open in distal direction.

According to at least one embodiment, the rotation of the movable member by the predetermined angle with respect to the protection member results in a transition of the engagement of the coupling features from the first section into the second section. In other words: In the initial state, the first section of the coupling feature with the two sections is engaged with the other coupling feature. The rotation of the movable member by the predetermined angle results in a disengagement of the first section of the coupling feature with the other coupling feature and an engagement of the second section of the coupling feature with the other coupling feature. Particularly, the second section of the coupling feature is arranged behind the first section in the first rotational direction.

According to at least one embodiment, the movable member comprises a second coupling feature. For example, the second coupling feature is located in the region of the proximal end of the movable member. The second coupling feature may be a flexible or resilient arm with a protrusion, e.g. a radially outwardly projecting protrusion. The arm may be oriented in axial direction. A proximal end of the arm may be displaceable in radial direction, a distal end of the arm may be axially and/or rotationally and/or radially fixed to the rest of the moveable member, e.g. a main body thereof.

According to at least one embodiment, the housing element comprises a coupling feature. For example, the coupling feature of the housing element is a recess in the housing element.

According to at least one embodiment, the coupling feature of the housing element and the second coupling feature of the movable member are configured to engage in order to provide a coupling between the movable member and the housing element which prevents a movement of the movable member in distal direction, preferably also in proximal direction.

According to at least one embodiment, the drug delivery device is configured such that the coupling feature of the housing element and the second coupling feature of the movable member engage when, starting from the initial state, the protection member with the movable member coupled thereto is moved in proximal direction. For example, the coupling features engage only when the protection member is in the retracted position and/or after rotation of the moveable member by the predetermined angle. When engaged, the protrusion of the arm may project into the recess.

According to at least one embodiment, the drug delivery device is configured such that, in the released state, the transfer member moves axially with respect to the housing element. For example, the transfer member moves axially until it hits an end-stop, e.g. a proximal end-stop, of the drug delivery device. The end-stop may be formed by the housing element or by another element or member axially fixed with respect to the housing element. For example, the transfer member moves by at least 1 mm or at least 5 mm in axial direction, e.g. proximally. Preferably, the transfer member moves axially and/or rotationally during the axial movement of the plunger rod.

According to at least one embodiment, in the released state and after hitting the end-stop, the transfer member rotates or continues to rotate. A further axial movement may be prevented by the end-stop. For example, after hitting the end-stop, the transfer member rotates by at least 360°.

According to at least one embodiment, in the released state, the transfer member moves in proximal direction. In this case, the end-stop may be provided in the region of the proximal end of the drug delivery device.

According to at least one embodiment, the end-stop comprises a friction reduction element. Additionally or alternatively, the proximal end of the transfer member may comprise a friction reduction element.

According to at least one embodiment, a low friction interface is formed between the friction reduction elements of the transfer member and the end-stop.

According to at least one embodiment, at least one of the friction reduction elements is a tapering protrusion. Particularly, the protrusion tapers in direction of the respective other friction reduction element. The protrusion may have the shape of a cone. For example, the friction reduction element of the end-stop is a tapering protrusion.

According to at least one embodiment, the other one of the friction reduction elements is an indentation. The friction reduction element being the protrusion may project into the indentation when the transfer member hits the end-stop. The indentation may be formed by a concave surface at the proximal end of the transfer member.

According to at least one embodiment, the indentation and/or the protrusion are rotationally symmetric, preferably circular symmetric, with respect to the rotational axis of the transfer member and/or the longitudinal axis.

According to at least one embodiment, the energy member is a drive spring, particularly a torsion drive spring, connected to the transfer member at a first connection point and connected to the housing element at a second connection point. The connection of the drive spring to the transfer member and/or the housing element is preferably irreleasable or permanent. That is to say, the connection cannot be released without destroying the connection or the connection is present in every state of the drug delivery device.

According to at least one embodiment, during axial movement of the transfer member, the first connection point and the second connection point are axially moved with respect to each other. Particularly, the first connection point is moved with respect to the second connection point in proximal direction, when the transfer member moves proximally, e.g. in the released state.

According to at least one embodiment, the drug delivery device comprises a housing. The housing element may be fixed to the housing or integrated in the housing. The housing is preferably axially and rotationally, preferably also radially, fixed with respect to the housing element. The housing element may be part of the housing, e.g. integrally formed with the housing, or may be a separate element. The housing may comprise or consist of plastic and/or may be formed in one piece. The housing may be hollow and/or elongated and/or hollow cylindrically-shaped. The housing may be a sleeve. The housing may be configured to hold or receive a medicament container, e.g. a syringe. The housing may be configured to hold the medicament container such that it is axially and/or rotationally and/or radially fixed with respect to the housing. The housing element and/or the energy member and/or the plunger rod and/or the transfer member and/or the movable member may be received in the housing, i.e. circumferentially surrounded by the housing.

According to at least one embodiment, the drug delivery device comprises the medicament container. The medicament container may comprise a needle. The medicament container may be received in the housing, i.e. circumferentially surrounded by the housing. The needle may form the distal end of the medicament container. The medicament container may be located distally with respect to the transfer member and/or the plunger rod and/or the energy member and/or the movable member and/or the transfer member, especially in the initial state. The medicament container may be arranged axially and/or rotationally and/or radially fixed with respect to the housing, i.e. it is not moved with respect to the housing during the intended usage of the drug delivery device. The medicament container may be a syringe, e.g. a pre-filled syringe. An end of the container opposite the needle may be sealingly closed by a movable member, e.g. a stopper or piston. The medicament container may comprise a drug or medicament, e.g. a liquid drug or medicament. The drug delivery device may be configured to empty the medicament container when released. In other words, the medicament container may comprise medicament in an amount sufficient for just one drug delivery operation. The drug delivery operation may be performed when the drug delivery device has been switched into the released state. The drug delivery device may be a single use device and/or a disposable device.

According to at least one embodiment, the protection member is telescopically coupled to the housing and axially movable with respect to the housing between the extended position, e.g. a position in which the needle is covered by the protection member, and the retracted position, e.g. a position in which the needle is exposed. In the retracted position, the needle can be pierced into tissue of a body. The protection member may, in particular, be a needle shroud.

According to at least one embodiment, the medicament container comprises a stopper. The stopper may seal the medicament container in proximal direction. In the released state of the drug delivery device, a distal end of the plunger rod may abut against the stopper and may, driven by the energy member, push the stopper in distal direction. The movement of the stopper in distal direction may result in the drug in the medicament container to be pressed through the needle out of the drug delivery device.

According to at least one embodiment, in the initial state, the plunger rod is axially spaced from the stopper. Thus, in the released state, the plunger rod first moves in distal direction before it hits the stopper and then it pushes the stopper in distal direction. The axial movement of the transfer member preferably starts simultaneously with the axial movement of the plunger rod. Alternatively, the axial movement of the transfer member may only start with or after the plunger rod hits the stopper.

According to at least one embodiment, the movement of the stopper may start with a delay compared to the start of the movement of the transfer member and/or the plunger rod. For example, the transfer member first moves in the first direction and/or axially for a certain distance before the stopper starts to move.

According to at least one embodiment, the drug delivery device comprises a shroud spring. The shroud spring may be coupled to the protection member and the housing element or the housing of the drug delivery device. The shroud spring may be configured such that it induces a restoring force acting in distal direction on the protection member when the protection member is moved from the extended position towards the retracted position.

According to at least one embodiment, in the retracted position of the protection member, the shroud spring biases the protection member in distal direction. Particularly, the shroud spring forces the protection member in the retracted position to automatically move in distal direction towards or into the extended position.

The drug delivery device may be used as follows: First, the drug delivery device is in the initial state. Then, the distal end of the drug delivery device is pressed against a skin region of a body, e.g. a human body. At this state, the distal end of the drug delivery device may be formed by a distal end of the protection member. This forces the protection member to move from the extended position into the retracted position. The movable member is moved in proximal direction as well due to the coupling to the protection member. The movement in proximal direction biases the shroud spring and the biased shroud spring biases the protection member in distal direction with respect to the housing. In the retracted position, the drug delivery device switches from the initial state into the released state. Before or when reaching the retracted position, the movable member is rotated by the predetermined angle, e.g. in the first rotational direction, thereby decoupling from the protection member in distal direction. In the released state, the drug is delivered, e.g. injected into the tissue of the body. Afterwards, the distal end of the drug delivery device may be removed from the skin. The shroud spring forces the protection member to move in distal direction, e.g. back into the extended position. The movable member does not follow this movement as it is decoupled in distal direction from the protection member and optionally axially coupled to the housing element.

Hereinafter, the drug delivery device described herein will be explained in more detail with reference to drawings on the basis of exemplary embodiments. Same reference signs indicate same elements in the individual figures. However, the size ratios involved are not necessarily to scale, individual elements may rather be illustrated with exaggerated size for a better understanding.

### BRIEF DESCRIPTION OF THE FIGURES

FIGS. 1 to 6 show a first exemplary embodiment of the drug delivery device in different views.

FIG. 7 to 12 show different positions during usage of the drug delivery device according to the first exemplary embodiment.

FIG. 13 shows the drug delivery device according to the first exemplary embodiment in an exploded view.

FIGS. 14 to 16 show subassemblies of the drug delivery device according to the first exemplary embodiment in more detail.

FIGS. 17 to 22 show a second exemplary embodiment of the drug delivery device in different views.

FIGS. 23 and 24 show subassemblies of the drug delivery device according to the second exemplary embodiment in exploded views.

FIGS. 25 to 27 show a part or arrangement of the drug delivery device according to the first and second exemplary embodiment in different positions during usage for illustrating an exemplary embodiment of a drive mechanism.

FIGS. 28 to 33 show sections of the drug delivery device according to the first and second exemplary embodiment in different positions during usage for illustrating an exemplary embodiment of a first locking mechanism and the release of the first locking mechanism.

FIGS. 34 to 38 show sections of the drug delivery device according the first and second exemplary embodiment in different positions during usage for illustrating a first exemplary embodiment of a third locking mechanism.

FIGS. 39 and 40 show sections of the drug delivery device in different positions during usage for illustrating a second exemplary embodiment of the third locking mechanism.

FIGS. 41 and 42 show sections of the drug delivery device according to the first and second exemplary embodiment in different positions during usage for illustrating an exemplary embodiment of a drop protection mechanism.

FIG. 43 shows the different subassemblies of the drug delivery device according to the first exemplary embodiment and a step during assembling a drug delivery device.

FIGS. 44 to 46 show sections of the front subassembly of the drug delivery device according to the first exemplary embodiment.

FIGS. 47, 48 and 50 to 53 show different positions in an exemplary embodiment of a method for assembling the drug delivery device according to the first exemplary embodiment.

FIG. 49 shows an isolated drive spring holder of the drug delivery according to the first and second exemplary embodiments.

FIGS. 54 to 56 show an exemplary embodiment of a feedback mechanism in different positions.

FIGS. 57 to 62 show a third exemplary embodiment of a drug delivery device in different views.

FIG. 63 shows the drug delivery device according to the third exemplary embodiment after usage.

FIG. 64 shows different subassemblies of the drug delivery device according to the third exemplary embodiment.

FIGS. 65 and 66 show the subassemblies of the drug delivery device according to the third exemplary embodiment in exploded views.

FIGS. 67 to 70 show sections of the drug delivery device according to the third exemplary embodiment in different positions during usage for illustrating a locking mechanism.

FIGS. 71 to 73 show different positions during assembling the drug delivery device according to the third exemplary embodiment.

### DETAILED DESCRIPTION

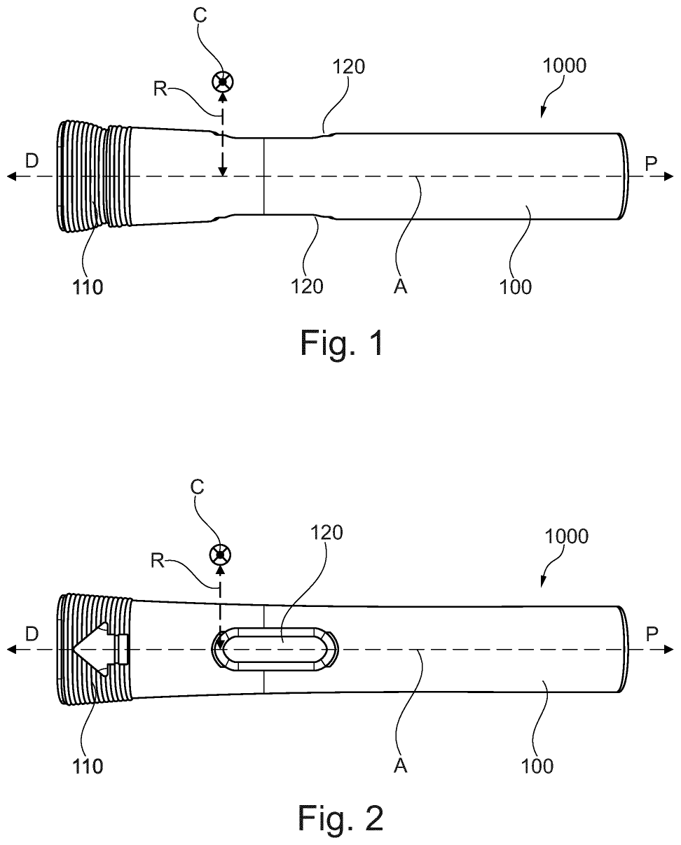

1. First exemplary embodiment of a drug delivery device FIGS. 1 and 2 show side views of a first exemplary embodiment of the drug delivery device 1000. FIG. 1 shows a first view of the drug delivery device 1000 and FIG. 2 shows a second view in which the device 1000 is rotated by 90° around a longitudinal axis A compared to the first view.

FIGS. 1 and 2 also indicate the coordinate system used herein for specifying positions of members or elements or features. The distal direction D and proximal direction P run parallel to the longitudinal axis A. The longitudinal axis A is a main extension axis of the device 1000. The radial direction R is a direction perpendicular to the longitudinal axis A and intersecting with the longitudinal axis A. The azimuthal direction C, also referred to as angular direction or rotational direction, is a direction perpendicular to the radial direction R and to the longitudinal axis A. The different directions and axes will not be indicated in every of the following figures in order to increase the clarity of the figures.

The drug delivery device 1000 according to the first exemplary embodiment is an auto-injector. The auto-injector 1000 comprises a housing 100. A cap 110 is removably attached or coupled to the housing 100 at a distal end of the housing 100. The housing 100 may be formed in one piece and may extend from the cap 110 to the proximal end of the auto-injector 1000. The housing 100 is a cylindrically-shaped sleeve.

As can be further seen in FIGS. 1 and 2, the housing 100 comprises windows 120 through which a medicament container inside the housing 100 can be investigated. For example, the fill level of the drug inside the medicament container or the advancement of a stopper in the medicament container or the drug clarity or the degradation of the drug can be observed through the windows 120.

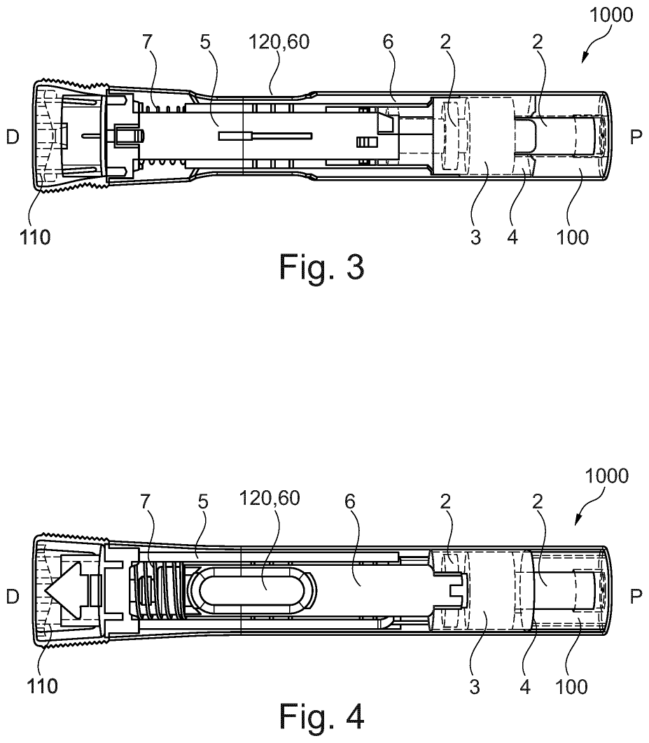

FIGS. 3 and 4 show the auto-injector 1000 in the same views as in FIGS. 1 and 2, but now the cap 110 and the housing 100 are indicated semi-transparent so that further details of the auto-injector 1000, which are normally completely surrounded and hidden by the housing 100 and the cap 110, are visible. It can be seen that the auto-injector 1000 further comprises a transfer member 2, also referred to as moveable member 2 or drive member 2, respectively, in the form of a rotating collar 2, an energy member 3 in the form of a torsion drive spring 3, particularly a spiral torsion drive spring (also commonly referred to as clock spring or power spring), and a housing element 4 in the form of a drive spring holder 4.

The drive spring holder 4 is fixed to the housing 100 so that the drive spring holder 4 can neither be rotated nor axially nor radially moved with respect to the housing 100. For example, the drive spring holder 4 is fixed with help of clips (not shown) to the housing 100. Alternatively, the drive spring holder 4 may be part of the housing 100, e.g. integrally formed with the housing 100. The drive spring holder 4 is received in the housing 100. The housing 100 circumferentially completely surrounds the drive spring holder 4.

The torsion drive spring 3 is connected to the drive spring holder 4 at a connection point. At a further connection point, the torsion drive spring 3 is connected to the rotating collar 2. The rotating collar 2 is arranged axially and rotationally movable with respect to the drive spring holder 4. The torsion drive spring 3 circumferentially surrounds a portion of the rotating collar 2. When the torsion drive spring 3 is biased, it induces a torque onto the rotating collar 2. This torque results in a rotation of the rotating collar 2 with respect to the drive spring holder 4, if the rotating collar 2 is not prevented from rotating by a locking mechanism (see explanations further down below). The rotational axis of the rotating collar 2 may define or coincide with the longitudinal axis A.

The auto-injector 1000 further comprises a release member 5 or protection member 5, respectively, in the form of a needle shroud 5 and a medicament container holder 6 in the form of a syringe holder 6. The syringe holder 6 may be axially and preferably also rotationally fixed with respect to the housing 100. The syringe holder 6 is configured to hold a syringe. The syringe holder 6 comprises windows 60 which overlap/are aligned with the windows 120 in the housing 100. In this way, the syringe or medicament container can be observed through the windows 60, 120.

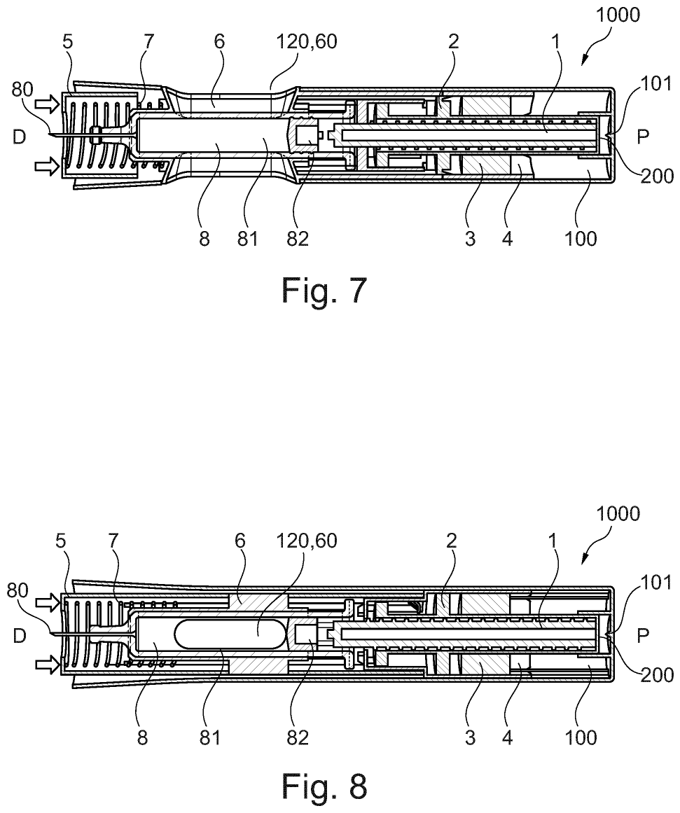

The needle shroud 5 is arranged axially movable with respect to and telescopically coupled to the housing 100 or the drive spring holder 4, respectively. Particularly, the needle shroud 5 can be moved from an extended position, which is the position shown in FIGS. 3 and 4, in the proximal direction P, into a retracted position (see FIGS. 7 and 8). This will be explained in more detail further below.

The needle shroud 5 and the syringe holder 6 are moveably coupled to each other via a shroud spring 7. One end of the shroud spring 7 is connected to the syringe holder 6 and the other end of the shroud spring 7 is connected to the needle shroud 5. The coupling is such that a movement of the needle shroud 5 in the proximal direction P with respect to the syringe holder 6 results in a compression of the shroud spring 7 inducing a force onto the needle shroud 5 pointing in distal direction D.

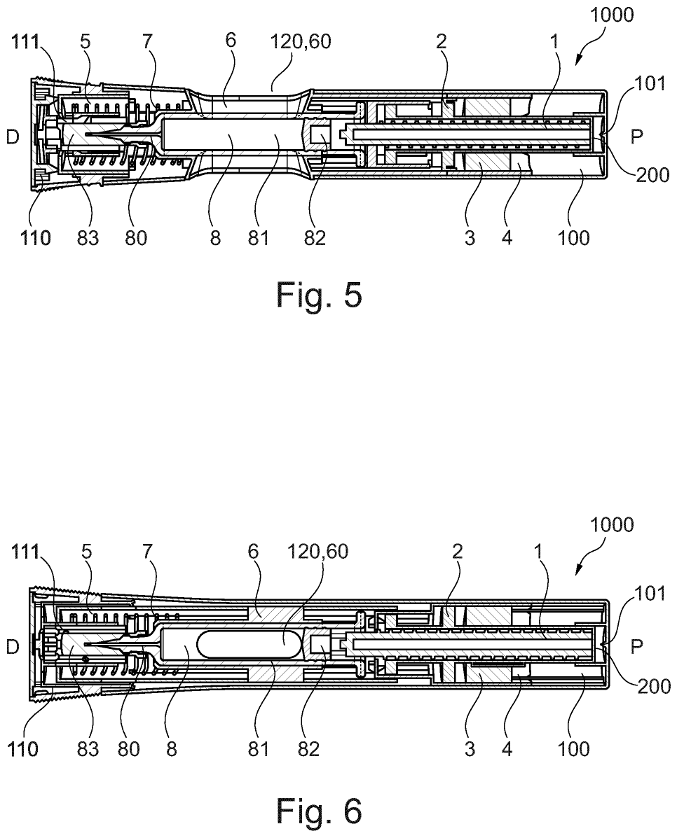

FIGS. 5 and 6 show the auto-injector 1000 in two cross-sectional views, the views again being rotated by 90° with respect to each other around the longitudinal axis. The cutting plane comprises the longitudinal axis A. In this view, it can be seen that the auto-injector 1000 further comprises a plunger rod 1. The plunger rod 1 is arranged inside the rotating collar 2 and is circumferentially surrounded by the rotating collar 2. Only a small portion of the plunger rod 1 (less than 50% of its length) projects out of the rotating collar 2 in distal direction D. In proximal direction P, the rotating collar 2 is closed and the plunger rod 1 does not project beyond the proximal end of the rotating collar 2. The plunger rod 1 is longer, measured along the longitudinal axis A, than the rotating collar 2.

The housing 100, the housing element 4, the plunger rod 1, the rotating collar 2, the needle shroud 5, the syringe holder 6 and the cap 110 may all comprise or consist of plastic. All these members may each be formed in one piece. The drive spring 3 and the shroud spring 7 may comprise or consist of a metal, e.g. steel.

It can be seen in FIGS. 5 and 6 that a medicament container 8, in the present case a syringe 8, is arranged in the syringe holder 6. This syringe 8 may be arranged axially and/or rotationally and/or radially fixed with respect to the syringe holder 6 and/or with respect to the housing 100. The syringe 8 comprises a cartridge 81 filled with a drug, a needle 80 and a stopper 82. The needle 80 is arranged at a distal end of the syringe 8. The stopper 82 seals the cartridge 81 in proximal direction P. When moving the stopper 82 in the distal direction D, the drug stored in the cartridge 81 is pressed out of the syringe 8 through the needle 80.

In FIGS. 5 and 6 it can be further seen that the needle 80 is covered by a needle shield 83 which encapsulates the needle 80 and projects beyond the needle 80 in distal direction D. The needle shield 83 may be formed of a rubber material. The cap 110 is connected to a grabber 111. The grabber 111 is retained within the cap 110 with a one or more bosses. The grabber 111 is coupled with the needle shield 83. The grabber 111 may be formed of a metal and may comprise barbs, which engage into the material of the needle shield 83.

When the cap 110 is removed from the housing 100, the grabber 111 pulls of the needle shield 83 from the needle 80. Afterwards, the needle 80 is circumferentially only surrounded by the retractable needle shroud 5.

FIGS. 7 and 8 show the auto-injector 1000 in the two cross-sectional views during usage. A first position during usage is shown in which the cap 110, the grabber 111 and the needle shield 83 have been removed from the housing 100. The needle shroud 5 projects from the housing 100 in distal direction D.

In the position of FIGS. 7 and 8, the distal end of the auto-injector 1000 formed by the needle shroud 5 may be pressed against a body, e.g. a human body. As a consequence of that, the needle shroud 5 moves from its extended position in the proximal direction P with respect to the housing 100. This results in the needle 80 being exposed and projecting in distal direction D beyond the needle shroud 5 so that it can now pierce or is already pierced into the tissue of the body.

In the position of FIGS. 7 and 8, the auto-injector 1000 is still in a first locked state, also referred to as pre-released state or initial state, (like in the previous figures), in which the torsion drive spring 3 is biased and induces a torque onto the rotating collar 2. A first locking mechanism (also referred to as first rotation-locking mechanism), however, prevents the rotating collar 2 from a rotational movement. The first locking mechanism will be explained in more detail further below.

In the first locked state, a proximal end of the rotating collar 2 is axially spaced from a proximal end-stop of the housing 100. This allows an axial movement of the rotating collar 2 in proximal direction P. Moreover, in the first locked state, a distal end of the plunger rod 1 is axially spaced from the stopper 82 of the syringe 8. Thus, the plunger rod 1 can axially move in the distal direction D for a predetermined distance before hitting the stopper 82.

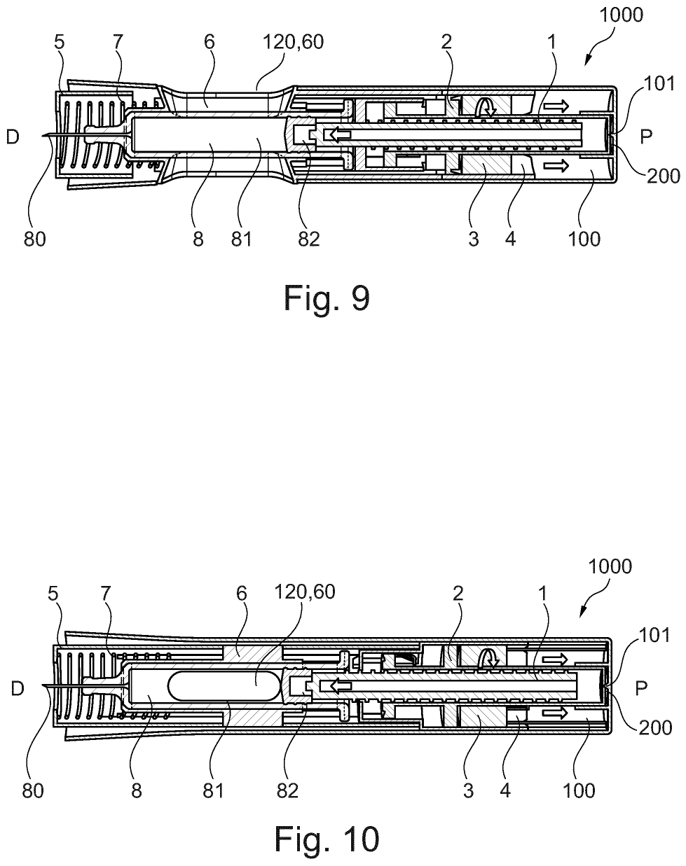

FIGS. 9 and 10 show the two cross-sectional views of the auto-injector 1000 in a second position during usage. The auto-injector 1000 is now in a released state. The needle shroud 5 has been further moved in the proximal direction P into a retracted position. This has released the first locking mechanism so that the rotating collar 2 was no longer prevented from rotating.

The torque induced by the torsion drive spring 3 onto the rotating collar 2 forces the rotating collar 2 to rotate in a first rotational direction (clockwise or counterclockwise). A drive mechanism, which will be explained in more detail further below, has converted the rotation of the rotating collar 2 into an axial movement of the plunger rod 1 in the distal direction D. After having moved the predetermined distance in the distal direction D, the plunger rod 1 has hit the stopper 82 of the syringe 8 and can now push the stopper 82 in distal direction D which results in the drug in the cartridge 81 being pressed out through the needle 80 into the tissue.

As indicated in FIGS. 9 and 10, the rotating collar 2 does not only rotate but also moves in proximal direction P until the proximal end of the rotating collar 2 hits the proximal end-stop of the housing 100. The end-stop comprises is a protrusion 101 which tapers in distal direction D. The protrusion 101 may be a cone. The proximal end of the rotating collar 2 comprises an indentation 200. For example, the surface of the proximal end of the rotating collar 2 is concavely shaped. The protrusion 101 can penetrate into the indentation 200 when the proximal end of the rotating collar 2 hits the end-stop of the housing 100. The protrusion 101 and the indentation 200 may each be designed rotationally symmetric or circular symmetric with respect to the rotational axis of the rotating collar 2. In this way, a low friction interface is formed between the housing 100 and the rotating collar 2 so that a low friction rotation of the rotating collar 2 is enabled also when the proximal end of the rotating collar 2 abuts against the housing 100. Particularly, the radius at which the friction between the rotating collar 2 and the end-stop acts is approaching zero or is zero, therefore the resulting torque from the friction also tends to zero significantly reducing losses allowing a reduced spring force and/or enhance injection performance.

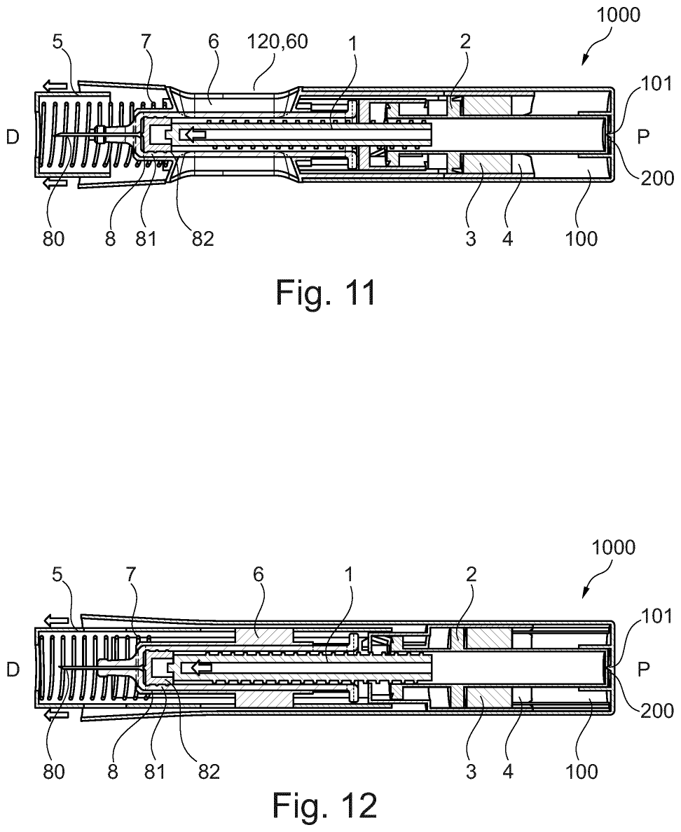

FIGS. 11 and 12 show two cross-sectional views of the auto-injector 1000 in a third position during usage. The torsion drive spring 3 has further induced torque onto the rotating collar 2, which, although abutting against the end-stop of the housing 100, has further rotated and has thereby forced the plunger rod 1 to move further in distal direction D. The plunger rod 1 has pushed the stopper 82 further in distal direction D so that a predetermined dose of the drug was supplied through the needle 80, e.g. into the tissue. Between the described first and third position, the rotating collar 2 has, e.g., rotated several times around its rotational axis.

In FIGS. 11 and 12, the auto-injector 1000 is in a third locked state or post-released state, in which the needle shroud 5 is again in its extended position so that it circumferentially surrounds the needle 80 and so that the needle 80 does no longer distally project beyond the needle shroud 5. The movement of the needle shroud 5 in the extended position happens automatically due to the force induced by the shroud spring 7 which has been compressed when moving the needle shroud 5 out of the extended position towards the retracted position.

In the third locked state of the auto-injector 1000 shown in FIGS. 11 and 12, the needle shroud 5 cannot be moved back into the retracted position due to a third locking mechanism which will be explained in more detail further below.

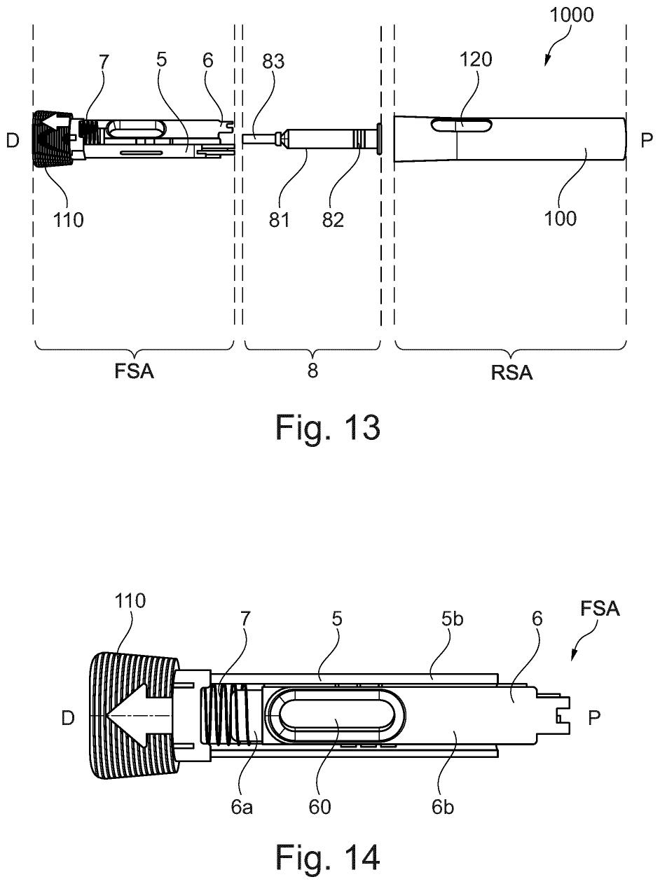

FIG. 13 shows the auto-injector 1000 of the previous figures in an exploded view. The auto-injector 1000 comprises a release subassembly FSA or front subassembly FSA, respectively, a drive subassembly RSA or rear subassembly RSA, respectively, and the syringe 8. For assembling the auto-injector 1000, the syringe 8 is inserted into the front subassembly FSA or the rear subassembly RSA and afterwards the front subassembly FSA is inserted into the rear subassembly RSA. Assembling of the auto-injector 1000 will be explained in more detail further below.

FIG. 14 shows the front subassembly FSA in a more detailed side view. The syringe holder 6 comprises two elongated arms 6b which extend axially and which are spaced from one another along the angular direction. The needle shroud 5 also comprises two elongated arms 5b which extend axially and which are spaced from one another along the angular direction. The needle shroud 5 and the syringe holder 6 are inserted into each other such that the arms 5b of the needle shroud 5 are located between the arms 6b of the syringe holder 6 along the angular direction. Furthermore, it can be seen that the arms 6b of the syringe holder 6 project beyond the arms 5b of the needle shroud 5 in proximal direction P.

The distal end of the syringe holder 6 is formed by a distal portion 6a in the form of a cylindrically-shaped portion 6a. This portion 6a is configured to hold the shroud spring 7. The cylindrically-shaped portion 6a is inserted into the shroud spring 7 so that an edge of the syringe holder 6 abuts against the proximal end of the shroud spring 7. The shroud spring 7 circumferentially surrounds the cylindrically-shaped portion 6a of the syringe holder 6. The shroud spring 7 may be fixed to the cylindrically-shaped portion 6a, e.g. by a glue or a mechanical radial interference with the proximal coil of the shroud spring 7.

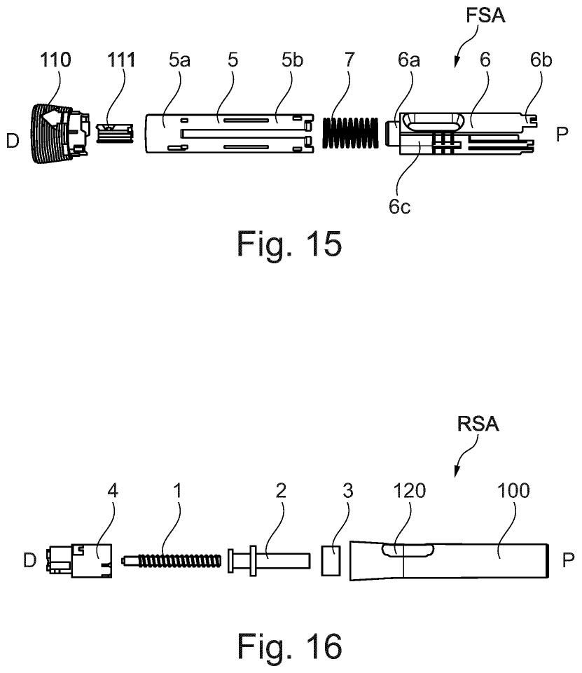

FIG. 15 shows the front subassembly FSA in an exploded view. It comprises the cap 110, the grabber 111, the needle shroud 5, the shroud spring 7 and the syringe holder 6. The needle shroud 5 also comprises a distal portion 5a in the form of a cylindrically-shaped portion 5a forming the distal end of the needle shroud 5. The cylindrically-shaped portion 5a is configured to hold the shroud spring 7. This cylindrically-shaped portion 5a is shaped as a hollow cylinder so that the shroud spring 7 can be inserted into this portion 5a and so that the distal end of the shroud spring 7 abuts against a bottom area of the cylindrically-shaped portion 5a. The shroud spring 7 may be fixed to the cylindrically-shaped portion 5a, e.g. by a glue or a mechanical radial interference with the distal coil of the shroud spring 7. In this way, the needle shroud 5, the shroud spring 7 and the syringe holder 6 are coupled such that a movement of the needle shroud 5 in proximal direction P with respect to the syringe holder 6 results in a compression of the shroud spring 7. The shroud spring 7 could also be held in place by a coupling/snap between the needle shroud 5 and the syringe holder 6 at the most extended positions, e.g. by the features 54 and 61 explained further below.

As can further be seen in FIG. 15, the syringe holder 6 comprises a support portion 6c, which is located proximally with respect to the cylindrically shaped portion 6a and which is located between the arms 6b and the cylindrically shaped portion 6a. After inserting the syringe holder 6 into the needle shroud 5, the arms 5b of the needle shroud 5 cover the support portion 6c, i.e. are located radially outwardly with respect to the support portion 6c.

FIG. 16 shows the rear subassembly RSA in an exploded view. The rear subassembly RSA comprises the housing 100, the torsion drive spring 3, the rotating collar 2, the plunger rod 1 and the drive spring holder 4. The drive spring holder 4, the rotating collar 2 and the housing 100 each have the form of a sleeve. When assembling the rear subassembly RSA, the plunger rod 1 is inserted into the rotating collar 2, the rotating collar 2 is inserted into the torsion drive spring 3 and is fixed to the torsion drive spring 3 at one connection point. The torsion drive spring 3 is inserted into the drive spring holder 4 and is connected to the drive spring holder 4 at a further connection point. The drive spring holder 4 is inserted into the housing 100.

### 2. Second Exemplary Embodiment of a Druq Delivery Device

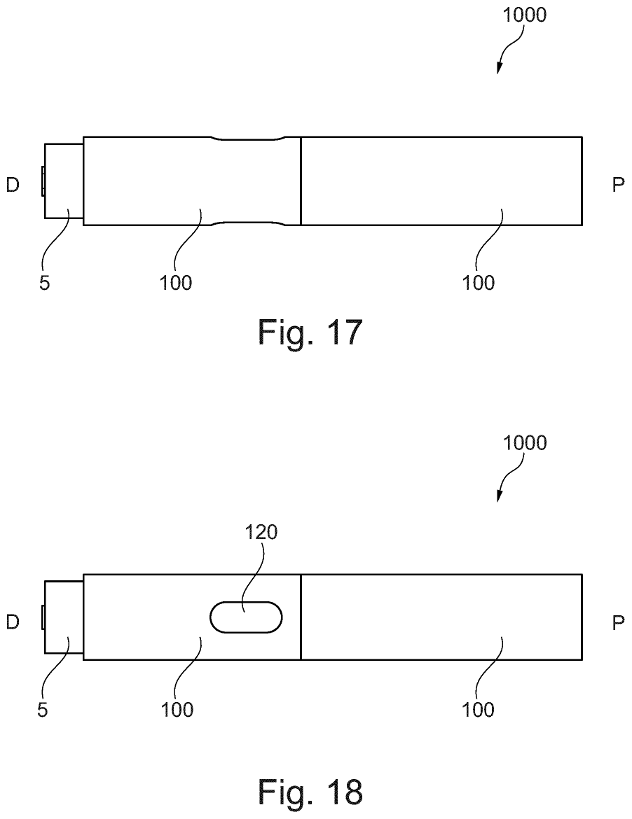

FIGS. 17 and 18 show a second exemplary embodiment of a drug delivery device 1000 which is again an auto-injector 1000. Like FIGS. 1 and 2, FIGS. 17 and 18 show the auto-injector 1000 in two different views rotated with respect to each other by 90° around the longitudinal axis A.

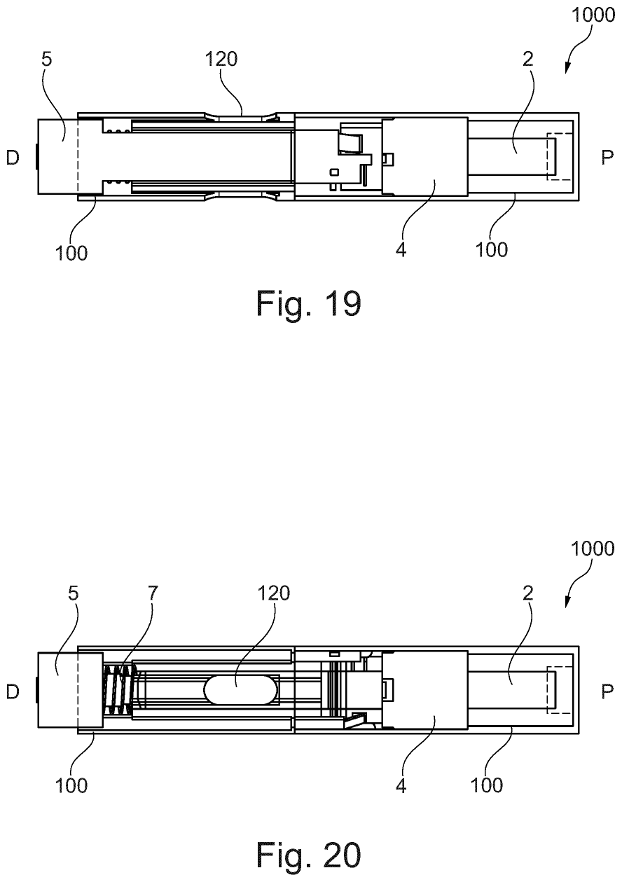

FIGS. 19 and 20 show the auto-injector 1000 of FIGS. 17 and 18 in the same rotated views but with a semi-transparent housing 100.

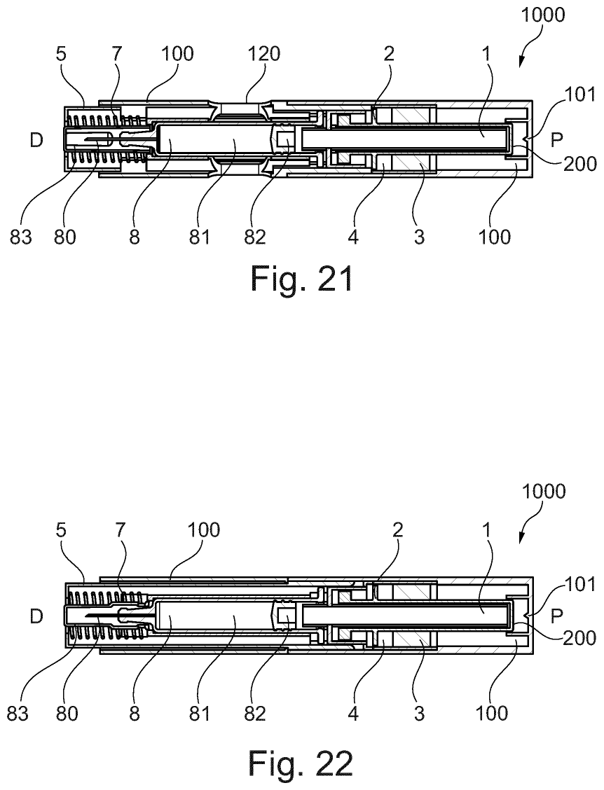

FIGS. 21 and 22 show the auto-injector 1000 of FIGS. 17 and 18 in the same rotated views but now in cross-sectional view with the crossing plane comprising the longitudinal axis.

One difference between the auto-injector 1000 according to the second exemplary embodiment and the auto-injector according to the first exemplary embodiment is that, in the second exemplary embodiment, the housing 100 now comprises two parts instead of one part. A first part forming the distal part of the housing 100 and a second part forming the proximal part of the housing 100. The two parts of the housing 100 are connected to each other, e.g. with help of clips (not shown). For example, the two parts of the housing 100 are fixed to each other such that they are neither axially nor rotationally nor radially movable with respect to each other.

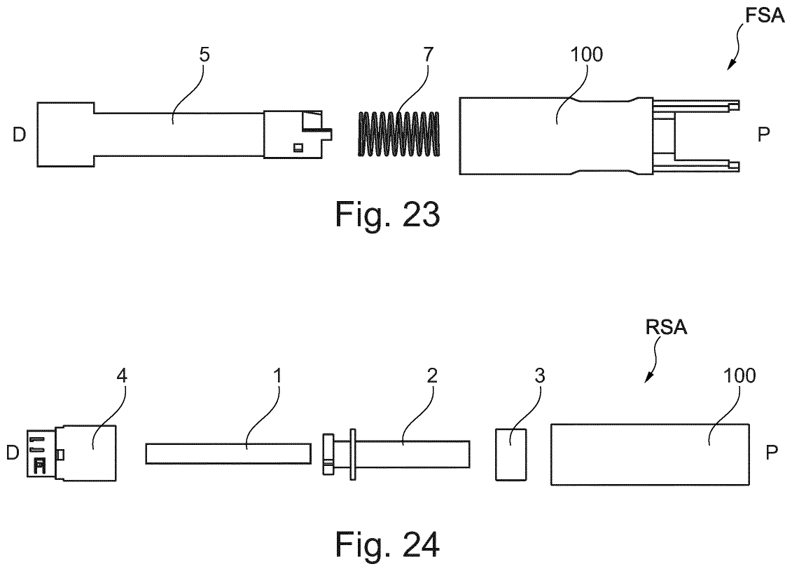

FIG. 23 shows a front subassembly FSA of the auto-injector 1000 according to the second exemplary embodiment in an exploded view. The first part of the housing 100 is assigned to the front subassembly FSA. The needle shroud 5 may be inserted into this first part of the housing 100. The shroud spring 7 is connected to the needle shroud 5 and the first part of the housing 100 so that a movement of the needle shroud 5 in proximal direction with respect to the first part of the housing 100 results in a compression of the shroud spring 7. In difference to the first exemplary embodiment, the auto-injector according to the second exemplary embodiment does not comprise a syringe holder with two arms spaced in angular direction. Instead of such a syringe holder, the first part of the housing 100 is configured to hold a medicament container, e.g. in an axially and/or rotationally fixed manner. The first part of the housing 100 circumferentially completely surrounds the needle shroud 5.

An exploded view of the rear subassembly RSA of the auto-injector 1000 according to the second exemplary embodiment is shown in FIG. 24. This rear subassembly is substantially identical to the rear subassembly RSA of the first exemplary embodiment. The second part of the housing 100 assigned to the rear subassembly RSA may be shorter than the housing 100 of the first exemplary embodiment.

### 3. Drive Mechanism

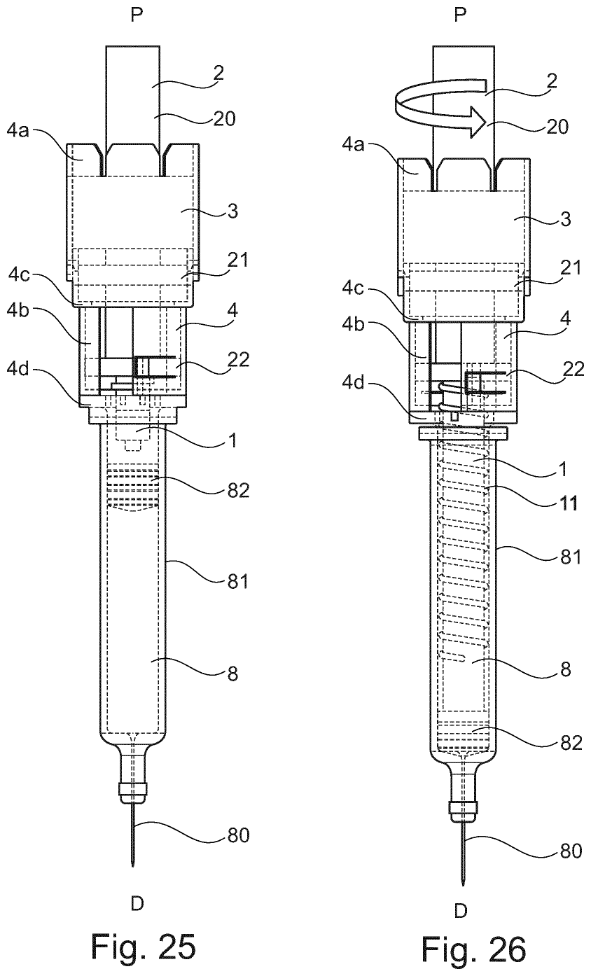

The conversion of the rotational movement of the rotating collar 2, induced by the torsion drive spring 3, into an axial movement of the plunger rod 1 (drive mechanism) is explained in more detail in the following in connection with FIGS. 25 to 27.

FIGS. 25 and 26 show a part or arrangement of the auto-injector 1000 of the first and second exemplary embodiment in different positions during usage. The shown part comprises the rear subassembly (the housing is not shown) and a syringe 8. In FIG. 25, the auto-injector 1000 is in the first locked state and in FIG. 26 the auto-injector is in the released state.

As can be seen in FIGS. 25 and 26, the drive spring holder 4 comprises two hollow sections 4a, 4b, which may both be hollow cylindrically-shaped. The two sections 4a, 4b are arranged behind each other along the longitudinal axis. The first section 4a is located more proximally and has a greater inner diameter and a greater outer diameter than the second section 4b.

The rotating collar 2 is received in the drive spring holder 4. A proximal end of the rotating collar 2 projects out of the drive spring holder 4 in proximal direction P. The rotating collar 2 comprises a shaft 20, and two portions 21, 22 with larger diameters than the shaft 20. The two portions 21, 22 are axially spaced from one another and are connected via the shaft 20. In this exemplary embodiment, the two portions 21, 22 are disc-shaped but other shapes might also be possible. The first portion 21 has a greater diameter than the second portion 22. The first portion 21 is located in the first section 4a of the drive spring holder 4 and the second portion 22 is located in the second section 4b of the drive spring holder 4. The diameters of the portions 21, 22 are substantially the same as the inner diameters of the assigned sections 4a, 4b but sufficiently smaller to allow a rotation of the rotating collar 2 with respect to the drive spring holder 4. Moreover, the diameter of the first portion 21 is greater than the inner diameter of the second section 4b which limits the axial movement of the rotating collar 2 in distal direction D.

As can be further seen in FIG. 25, in the first locked state, the second portion 22 is offset in proximal direction P from a second bottom ring 4d of the drive spring holder 4. Likewise, the first portion 21 is offset in proximal direction P from a first bottom ring 4c of the drive spring holder 4.

The torsion drive spring 3 is received in the first section 4a and is fixed to the first section 4a at a connection point. The rotating collar 2 is received in the torsion drive spring 3 so that the torsion drive spring 3 circumferentially surrounds the shaft 20 of the rotating collar 2 at a proximal side of the first section 21. The shaft 20 of the rotating collar 2 is connected to the torsion drive spring 3 at a further connection point. The first portion 21 is offset with respect to the torsion drive spring 3 in distal direction D. In the first locked state, shown in FIG. 25, the torsion drive spring 3 is biased and induces a torque onto the rotating collar 2. The rotating collar 2 is prevented from a rotation with help of the first locking mechanism explained further below.

The plunger rod 1 is received in the rotating collar 2. In the first locked state, a portion of the plunger rod 1 projects from the rotating collar 2 in distal direction D. The stopper 82 of the syringe 8 is offset from the distal end of the plunger rod 1 in distal direction D.

FIG. 26 shows the part or arrangement of the auto-injector in the released state. The first locking mechanism has been released so that the rotating collar 2 was no longer prevented from rotating. Due to the torque induced by the drive spring 3, the rotating collar 2 rotates in a first rotational direction (clockwise or anti-clockwise) inside the drive spring holder 4. The rotating collar 2 and the plunger rod 1 are operatively coupled via a threaded interface. In the present case, the plunger rod 1 comprises an external thread 11 and the rotating collar 2 comprises an internal thread (not visible) engaging with the external thread 11 of the plunger rod 1. The coupling via the threaded interface is such that the rotation of the rotating collar 2 in the first rotational direction is converted into a movement of the plunger rod 1 in distal direction D.

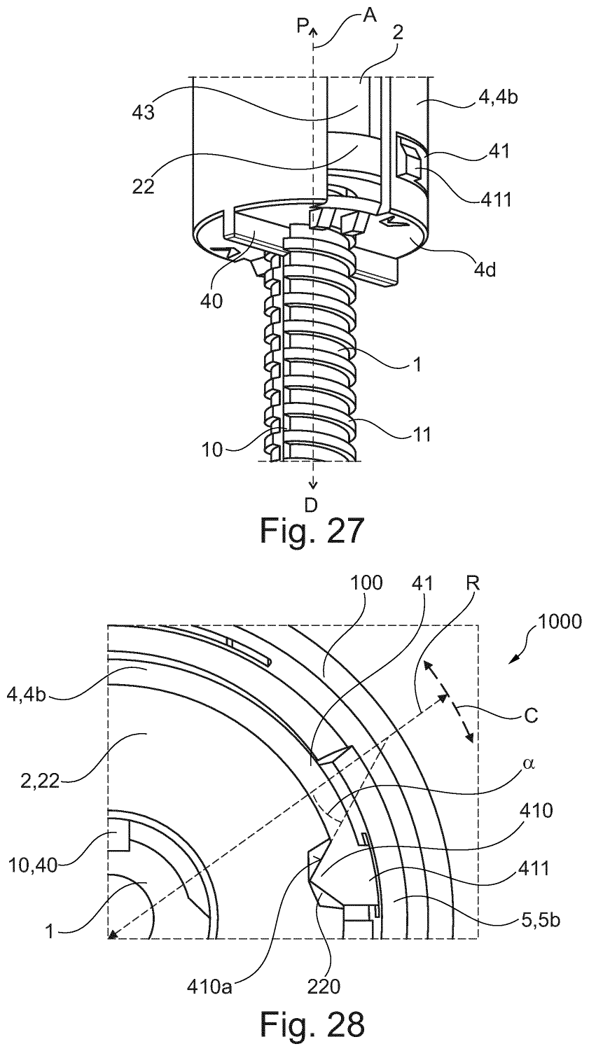

During the axial movement of the plunger rod 1 induced by the rotation of the rotating collar 2, the plunger rod 1 itself does not rotate. This is realized by a coupling between the plunger rod 1 and the drive spring holder 4 via a splined interface. This is further illustrated in FIG. 27 showing a three-dimensional view of the part/arrangement of the auto-injector. The splined interface is realized by protrusions 40 of the drive spring holder 4 projecting in distal direction D from the second bottom ring 4d and engaging with or projecting into grooves 10 of the plunger rod 1, respectively. The grooves 10 extend along the longitudinal axis A, i.e. run essentially parallel to the longitudinal axis A. The grooves 10 are arranged opposite each other on the plunger rod 1. Instead of two grooves, as shown in FIG. 27, one groove and one corresponding protrusion 40 may be sufficient. However, more than two grooves 10 and associated protrusions 40 may also be used.

In the exemplary embodiments, the splined interface is in close proximity to the threaded interface, e.g. with a distance of at most 1 cm or at most 0.5 cm. This is beneficial since the torque on the plunger rod 1 is resolved over a short distance reducing the stresses within the plunger rod 1. The plunger rod 1 is often a small member likely to deform.

As can be further seen in FIG. 26, the rotating collar 2 does not only rotate but also moves axially in the proximal direction P, as already mentioned before. The movement in proximal direction P preferably starts immediately when the rotation is started. In this way the needle shroud 5 may reextend upon premature removal from the skin. The break loose force of the stopper 82 is typically 5 N or more. The ability of the torsion drive spring 3 to resolve axial loads may be smaller than this.

In the released state, the plunger rod 1 pushes the stopper 82 in distal direction D until the stopper 82 hits against a bottom region of the cartridge 81. A further distal movement of the stopper 82 and the plunger rod 1 is then prevented. After movement of the plunger rod 1 and the rotating collar 2 is finished, a portion of the plunger rod 1 is still received in the rotating collar 2.

An example of the dimensions of the plunger rod 1 is as follows: The plunger rod 1 has a diameter of 8.0 mm and the pitch of the outer thread is 3.17 mm. The coefficient of friction is 0.3. The mean contact radius, i.e. the position of the threaded face from the central axis of the plunger rod 1, is 3.75 mm.

An example of the torsion drive spring is 3 as follows: The material is polished and blued SAE 1095 steel. The height of the torsion drive spring is 12.0 mm, the thickness of the material is 0.168 mm, the length is 840.749 mm, the outer diameter is 20.0 mm, the arbor diameter is 10.0 mm. The bending stress limit is 2000 N·mm⁻², the Youngs Modulus is 20000 N·mm⁻², the number of revolutions before being biased is 3.

In general, the following conditions for the torsion drive spring turned out to be advantageous: The arbor diameter is between 12 to 25 times the thicknesses of the material. The length is between 5000 to 15000 times the thickness. The area of the torsion drive spring 3 is half the area of the drive spring holder 4 (e.g. in the first section 4a)+−10%. The bending stress for tempered polished and blued SAE 1095 steel should not exceed 2000 MPa.

An example of the used syringe 8 might be as follows: The drug inside the cartridge 81 has a volume of 2 ml. The viscosity of the material is 50 cP at room temperature. The inner needle diameter is 0.29 mm. The inner cartridge diameter is 8.65 mm. The friction of the stopper 82 is 10 N. The stopper gap, i.e. the initial clearance between the proximal end of the stopper 82 and the distal end of the plunger rod 1, is 2 mm.

### 4. First Locking Mechanism and Release of the First Locking Mechanism

The previously mentioned first locking mechanism or first rotation-locking mechanism, respectively, and how it is released is described in further detail in the following in connection with the FIGS. 28 to 33.

FIG. 28 illustrates a cross-sectional view of the auto-injector 1000 of the first and second exemplary embodiment with the cutting plane being perpendicular to the longitudinal axis A and running through the second portion 22 of the rotating collar 2. As can be seen, the drive spring holder 4 comprises a displaceable element 41 in the form of a resilient arm (see also FIGS. 27 and 49). The resilient arm 41 is integrally formed with the drive spring holder 4 and is arranged in the second section 4b of the drive spring holder 4. The resilient arm 41 is oriented circumferentially, i.e. a main extension direction of the resilient arm 41 is along the angular direction C. One end of the resilient arm 41 is connected to the drive spring holder 4, the other end is free and movable in radial direction R.

The resilient arm 41 comprises a protrusion 410 projecting radially inwardly, i.e. in a radial direction pointing towards the longitudinal axis A. The protrusion 410 tapers radially inwardly. The protrusion 410 comprises a beveled surface 410a, which essentially runs parallel to the longitudinal axis A and which is tilted with respect to the radial direction R and with respect to the angular direction C. For example, the angle α between the beveled surface 410a and the radial direction R is at least 10° and at most 80°, preferably between 30° and 55°.

In the first locked state, shown in FIG. 28, the resilient arm 41 is in a first radial position in which the protrusion 410 engages or projects into a recess 220 of the second portion 22 of the rotating collar 2, respectively. In this way a rotation-lock interface is formed, coupling the resilient arm 41 and the rotating collar 2 and preventing the rotating collar 2 from a rotation.

The first radial position may be the relaxed position of the resilient arm 41 which it would occupy if no further forces pointing radially inwardly and radially outwardly were acting on the resilient arm 41. Alternatively, the resilient arm 41 may be biased in the first radial position, such that the first radial position is a stressed position of the resilient arm 41.

As long as the resilient arm 41 is in the first radial position in which the protrusion 410 projects into the recess 220, a rotation of the rotating collar 2 in the first rotational direction induced by the torsion drive spring 3 is prevented. However, the torque acting on the rotating collar 2 presses a surface of the second portion 22 delimiting the recess 220 against the beveled surface 410a of the protrusion 410 of the resilient arm 41. This results in a force trying to move the resilient arm 41 radially outwardly from the first radial position into a second radial position. In other words, the torque induced by the torsion drive spring 3 biases the resilient arm 41 radially outwardly. If a movement in radial outward direction would be allowed, the first locking mechanism would be released automatically and the auto-injector 1000 would transfer into the released state.

In the first locked state, an arm 5b of the needle shroud 5 is located at the height of, i.e. axially overlapping or aligned with, the resilient arm 41 and prevents the resilient arm 41 from moving radially outwardly out of and away from the first radial position. Indeed, the resilient arm 41 abuts against the needle shroud 5 in outward radial direction such that an outward radial movement is blocked. The resilient arm 41 comprises a further protrusion 411 which projects radially outwardly and which abuts against the needle shroud 5. An outward radial movement of the needle shroud 5 is prevented, e.g. by the housing 100 circumferentially surrounding the needle shroud 5.

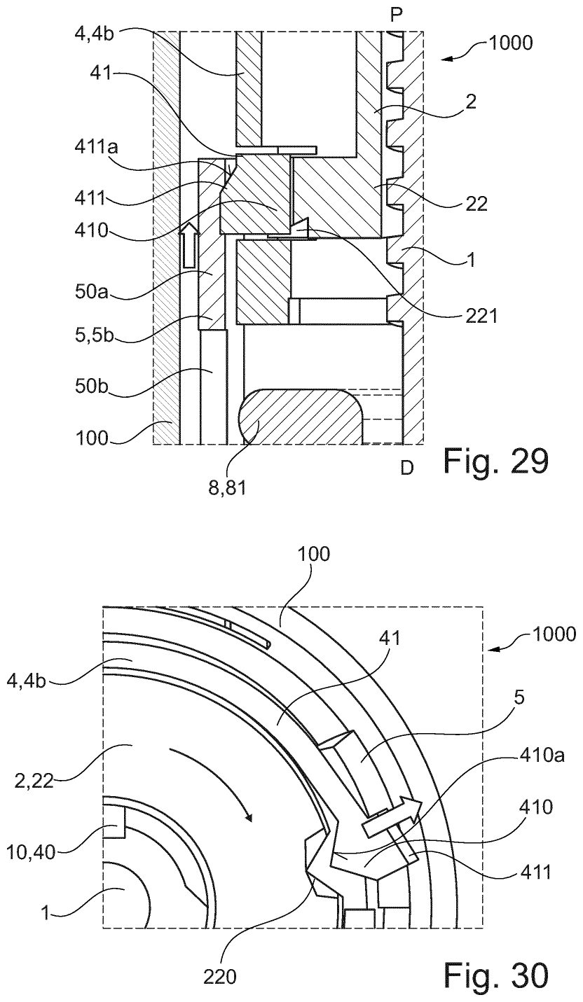

FIG. 29 shows a section of the auto-injector 1000 in the same state as in FIG. 28 but now in a cross-sectional view with the longitudinal axis lying in the cutting plane. There it can be seen that the arm 5b of the needle shroud 5 comprises a first section 50a, namely a wall portion, and a second section 50b, namely a recess, e.g. a cut-out. The recess 50b is offset in distal direction D with respect to the wall portion 50a. In the first locked state, the needle shroud 5 is in its extended position, in which the wall portion 50a blocks the outward radial movement of the resilient arm 41.

FIG. 29 further indicates that the needle shroud 5 can be moved from its extended position into a retracted position which would result in an overlap or alignment between the recess 50b and the resilient arm 41 in axial direction and rotational direction. Movement of the needle shroud 5 in proximal direction P requires a force, also called activation force, which includes the force needed to compress the shroud spring 7 and the friction force resulting from the resilient arm 41 being pressed against the needle shroud 5.

As an numerical example: Assuming a torque induced by the torsion drive spring 3 onto the rotating collar 2 of 102 Nmm, a radius at which the rotating collar 2 abuts against the protrusion 410 of 7.5 mm and an angle α of 39° would result in a force on the resilient arm 41 in radial direction of about 10.57 N. Assuming a friction coefficient of 0.3, the friction force would be about 3.17 N. Assuming further that the force for compressing the shroud spring 7 is about 6 N, the activation force would be about 9 N.

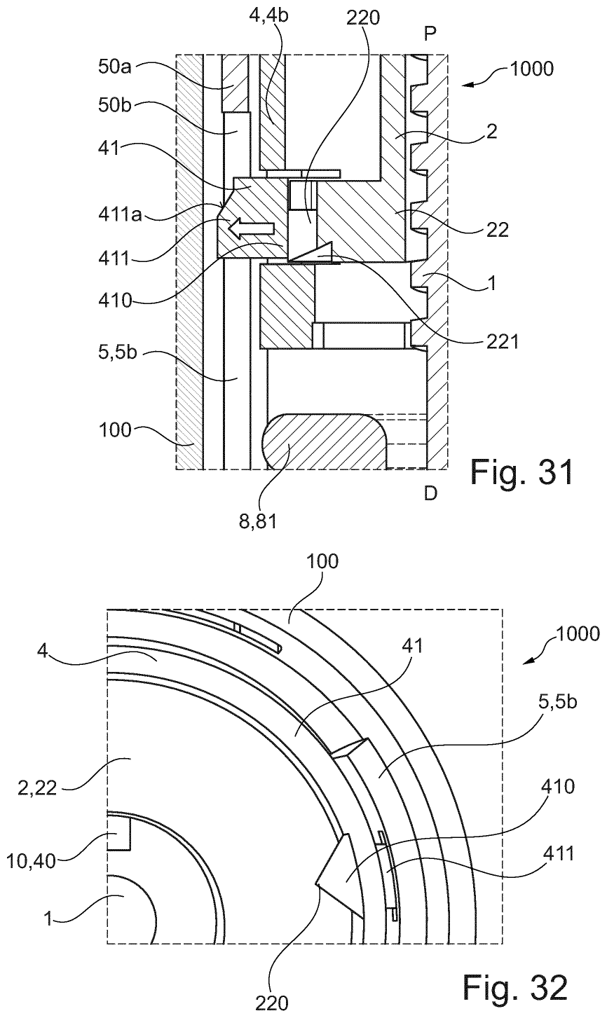

FIGS. 30 and 31 show sections of the auto-injector 1000 which corresponds to the sections shown in FIGS. 28 and 29. Now the needle shroud 5 has been moved in its retracted position (by overcoming the activation force). This movement releases the first locking mechanism so that the auto-injector 1000 is switched from the first locked state into the released state. As the recess 50b of the needle shroud 5 is now at the height of the resilient arm 41, the outward radial movement of the resilient arm 41 is no longer blocked. The resilient arm 41 automatically —induced by the torque on the rotating collar 2—leaves its first radial position and deflects into a second radial position in which the protrusion 410 no longer projects into the recess 220, thus the rotation-lock interface is resolved and the first locking mechanism is released. As a result of this, the rotation of the rotating collar 2 is no longer prevented. The rotating collar 2 starts to rotate (see FIG. 30) due to the force induced by the drive spring 3, thereby forcing the plunger rod 1 into an axial movement.

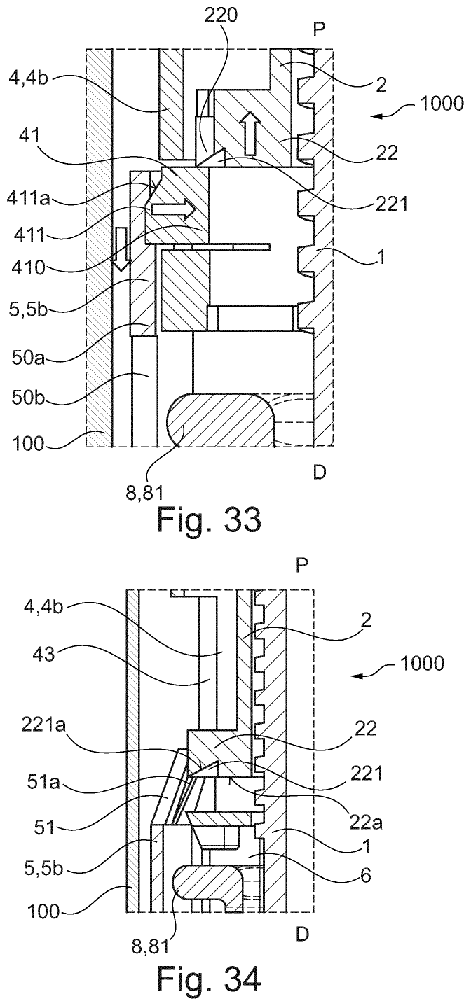

FIGS. 32 and 33 show sections of the auto-injector 1000 which corresponds to the sections shown in FIGS. 28 and 29. Now, the auto-injector 1000 is switched into a third locked state or post-released state. For example, the distal end of the auto-injector 1000 has been removed from the body so that the needle shroud 5 automatically moves from the retracted position back into the extended position induced by the shroud spring 7.

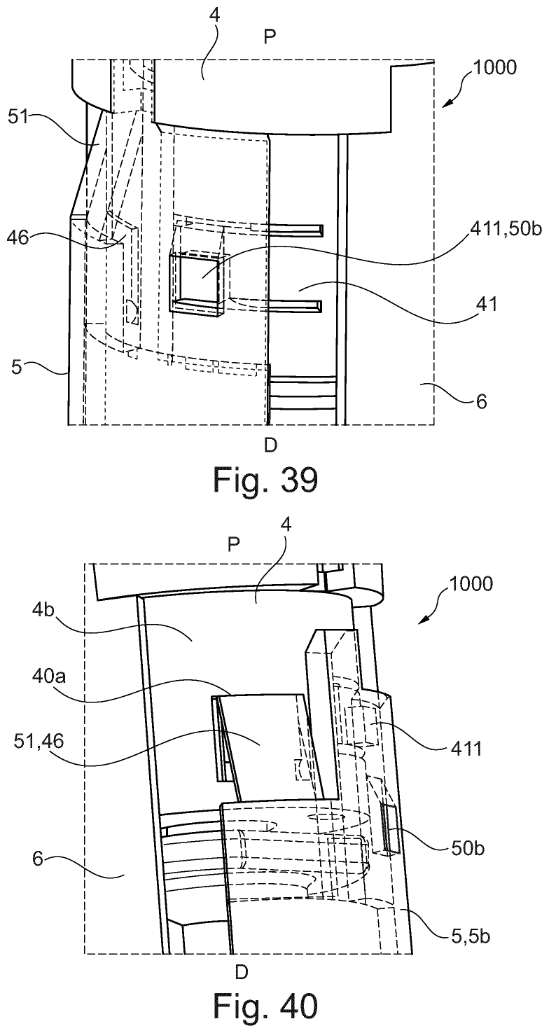

The protrusion 411 of the resilient arm 41 comprises a slide feature 411a in the form of a beveled surface 411a. The beveled surface 411a and the longitudinal axis may include, e.g., an angle between 10° and 80° inclusive. An edge of the needle shroud 5 delimiting the recess 50b in proximal direction P may contact this beveled surface 411a when the needle shroud 5 moves in distal direction D. Due to the beveled surface 411a, the resilient arm 41 is pushed radially inwardly when the edge hits the protrusion 411. In this way, the movement of the needle shroud 5 back into the retracted position is possible without the needle shroud 5 jamming up with the resilient arm 41. The slide feature may additionally or alternatively be formed in the needle shroud 5 (see FIGS. 39 and 40).

In the case the resilient arm 41 indeed abuts against the edge of needle shroud 5 when the needle shroud 5 is moved in distal direction D, the movement of the resilient arm 41 in inward radial direction is possible, since the rotating collar 2, particularly the second portion 22 of the rotating collar 2, has moved in proximal direction P. Thus, the second portion 22 is now offset in proximal direction P with respect to the resilient arm 41. For this reason, it is particularly beneficial if the rotating collar 2 moves in the proximal direction immediately when the plunger rod 1 starts to move in distal direction, i.e. before the plunger rod 1 hits the stopper 82. If the user lifts the auto-injector 1000 early from the skin, e.g. before the drug is started to be administered, the needle shroud 5 can then still move back in distal direction and the third locking mechanism explained below can be activated.

### 5. Third Locking Mechanism/Post-Released Locking Mechanism

A third locking mechanism or post-released locking mechanism, respectively, is described in further detail in the following in connection with the FIGS. 34 to 40.

FIGS. 34 to 38 illustrate a first exemplary embodiment of the third locking mechanism. This mechanism is configured to prevent the needle shroud 5 from being moved from the extended position into the retracted position after the drug has been delivered or after the autoinjector has once been activated. Thus, the risk of injuries due to an exposed needle may be reduced. This third locking mechanism may be used in all exemplary embodiments of the auto-injector 1000 described herein.

FIG. 34 shows again a cross-sectional view of a section of the auto-injector 1000 with the cutting plane comprising the longitudinal axis A. However, the cutting plane is rotated compared to what is shown in, e.g. FIG. 33 (see FIG. 38 for a perspective view). It can be seen in FIG. 34 that the arm 5b of the needle shroud 5 comprises a first stop feature 51 in the form of a displaceable element 51 which is located at the proximal end of the arm 5b. The displaceable element 51 is a resilient arm 51 which is integrally formed with the rest of the needle shroud 5 and, therefore, is axially and rotationally fixed to the rest of the needle shroud 5. Thus, the resilient arm 51 moves in axial direction when the needle shroud 5 is moved in axial direction.

As can be seen in FIG. 38, the resilient arm 51 is located on the same height as the wall portion 50a when seen along the longitudinal axis A and is arranged offset from the wall portion 50a in the angular direction C.

Simultaneously to extending in proximal direction P, the resilient arm 51 also extends radially inwardly, i.e. a main extension direction of the resilient arm 51 has a component along the proximal direction P and a component along the inward radial direction. Thus, a proximal end of the resilient arm 51 is located further radially inwardly than a distal end of the resilient arm 51. The proximal end of the resilient arm 51 is free and displaceable in the radial direction. The distal end of the resilient arm 51 is connected to the rest of the needle shroud 5. A kink is formed between the distal end of the resilient arm 51 and the rest of the needle shroud 5.

In FIG. 34, the auto-injector 1000 is in the first locked state (also referred to as initial state or pre-released state), in which a rotation of the rotating collar 2 is blocked by the first locking mechanism as described before. The resilient arm 51 is in a first radial position, which may be a biased position of the resilient 51. The resilient arm 51 is held in the first radial position and is prevented from moving radially inwardly by the second portion 22 of the rotating collar 2. In the present case, the drive spring holder 4 comprises a recess 43, namely a cut-out 43, into which the resilient arm 51 projects. The resilient arm 51 abuts against the second portion 22 in inward radial direction.

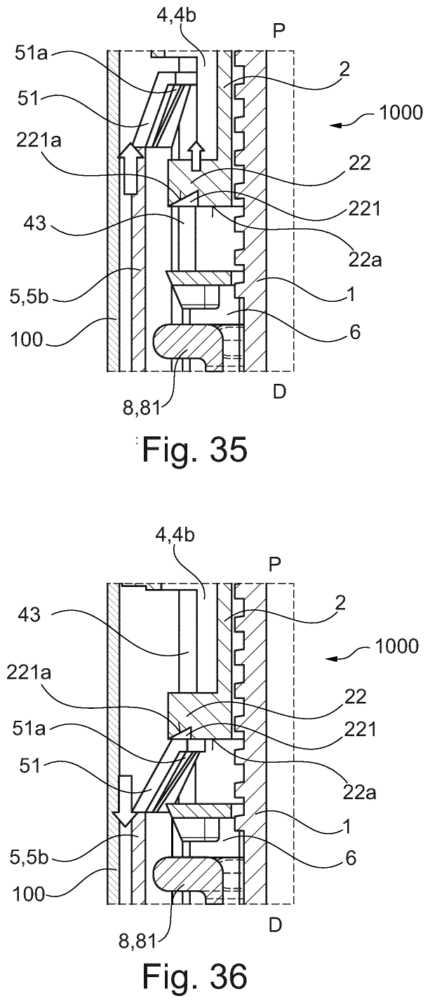

FIG. 35 shows a section of the auto-injector 1000 in a position during usage, when the needle shroud 5 is moved from its extended position into the retracted position so that the auto-injector 1000 switches into the released state. Together with the needle shroud 5, the resilient arm 51 has moved in proximal direction P such far that the second portion 22 of the rotating collar 2 does no longer hold the resilient arm 51 in the first radial position. This allowed the resilient arm 51 to move radially inwardly into a second radial position. In the released state of the auto-injector 1000 and the needle shroud 5 being in the retracted position, the resilient arm 51 is offset with respect to the second portion 22 in proximal direction P.

In the released state of the auto-injector 1000, the rotating collar 2 moves in proximal direction P from a nonlocking position into a locking position, as it is indicated in FIG. 35.

FIG. 36 shows a section of the auto-injector 1000 in a third locked state, also referred to as post-released state, which is a state after usage, i.e. after the drug has been dispensed. The third locked state is a state after the released state. In this third locked state, the needed shroud 5 is again in its extended position. As can be seen in FIG. 36, the second portion 22 has moved in proximal direction P such far that the resilient arm 51 is now offset in distal direction D with respect to the second portion 22 so that the second portion 22 can no longer hold the resilient arm 51 in the first radial position. Therefore, in the third locked state, the resilient arm 51 is in the second radial position. When trying to move the needle shroud 5 from the extended position towards the retracted position, the resilient arm 51 in the second radial position hits against a second stop feature 22a, namely a surface of the second portion 22 which runs essentially perpendicularly to the longitudinal axis and faces in distal direction D. This prevents a further movement of the needle shroud 5 in proximal direction P. For example, the auto-injector 1000 is configured such that, in the third locked state, the resilient arm 51 hits against the surface 22a of the second portion 22 when moving the needle shroud 5 in proximal direction P before the needle is exposed.

When the resilient arm 51 hits against the surface 22a of the second portion 22, a lock interface is formed between the resilient arm 51 and the surface 22a. For this purpose, a recess 221 or notch 221 is formed in the surface 22a which engages with the proximal end of the resilient arm 51 when the resilient arm 51 hits against the surface 22a. The recess 221 is delimited by a beveled surface 221a which is tilted with respect to the longitudinal axis A and the radial direction. For example, an angle between the beveled surface 221a and the longitudinal axis and/or the radial direction is between 10° and 80° inclusive. When the proximal end of the resilient arm 51 engages into the recess 221, the resilient arm 51 hits against the beveled surface 221a and slides along the beveled surface 221a thereby being forced to move radially inwardly. The recess 221 with the beveled surface 221a thus prevents the resilient arm 51 from sliding along the surface 22a in outward radial direction.

The surface 22a of the second portion 22 may circumferentially extend around the longitudinal axis and/or the rotational axis of the rotating collar 2 by at least 270° and may have a constant geometrical form along its extension along the angular direction. In this way, the functionality of the third locking mechanism is almost independent on how far the rotating collar 2 has rotated in the released state.

As can be further seen in FIGS. 34 to 36, the resilient arm 51 comprises a slide feature 51a in the form of a ramp 51a. During movement of the needle shroud 5 from the retracted position into the extended position, the ramp 51 hits against a proximal edge of the second portion 22. The ramp 51a is designed such that it forces the resilient arm 51 to slide along the edge of the second portion 22 so that the resilient arm 51 is pushed radially outwardly. This allows the resilient arm 51 to pass the second portion 22 without being jammed up with the second portion 22. After having passed the second portion 22 during movement towards the extended position, the resilient arm 51 springs back into the second radial position.

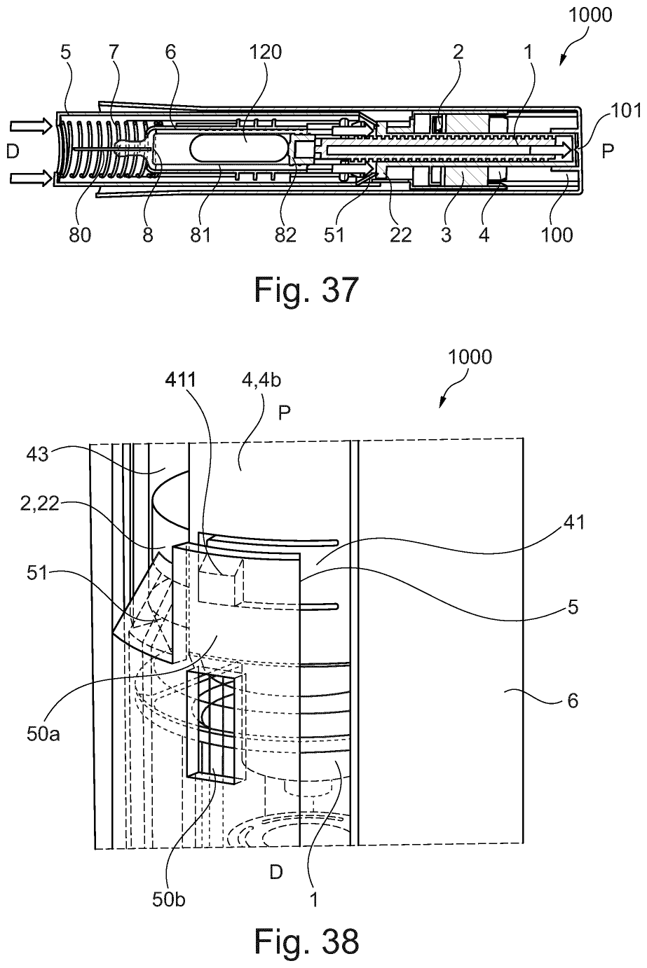

FIG. 37 shows the auto-injector 1000 in a cross-sectional view in the third locked state. As can be seen, the needle shroud 5 cannot be moved so far in proximal direction P that the needle 80 is exposed because the resilient arm 51 hits against the surface 22a of the second portion 22 before.

FIGS. 39 and 40 illustrate a second exemplary embodiment of the third locking mechanism. Also this exemplary embodiment of the third locking mechanism may be used in all exemplary embodiments of the auto-injector described herein.

The main difference to the first exemplary embodiment is that, in the third locked state of the auto-injector 1000, when moving the needle shroud 5 towards the retracted position, the resilient arm 51 does not hit against a stop feature axially fixed to the rotating collar 2 but against a stop feature 40a axially fixed to the drive spring holder 4. The stop feature 40a is formed by an edge of the drive spring holder 4. The edge 40a delimits a recess/cut-out in the drive spring holder 4 in proximal direction P.

A flap 46, which is axially fixed to the drive spring holder 4, e.g. integrally formed with the drive spring holder 4, partially fills this recess. A distal end of the flap 46 is connected to the drive spring holder 4 and a proximal end of the flap 46 is free and displaceable in radial direction. The proximal end of the flap 46 is spaced from the edge 40a by a small gap.

In the first locked state, when the needle shroud 5 is still in the extended position, the rotating collar 2, particularly the second portion 22 of the rotating collar 2, abuts against the flap 46 of the drive spring holder 4 in outward radial direction and holds the flap 46 in a first radial position, in which the flap 46 substantially terminates flush with the edge 40a in outward radial direction. The second portion 22 prevents the flap 46 from being displaced in the inward radial direction. On the other hand, the flap 46 abuts against the resilient arm 51 of the needle shroud 5. In the first radial position of the flap 46, the flap 46 holds the resilient arm 51 in its first radial position.

When now moving the needle shroud 5 in proximal direction P, the resilient arm 51 can pass the edge 40a without jamming up with the edge 40a, since the flap 46 terminates flush with the edge 40a and since the flap 46 is held in its first radial position by the second portion 22. Moving the needle shroud 5 further into its retracted position releases the first locking mechanism, the auto-injector 1000 switches from the first locked state into the released state and the rotating collar 2 together with the second portion 22 moves in proximal direction P into a locking position. The needle shroud 5 being in its retracted position is shown in FIG. 39.

When moving the needle shroud 5 back from its retracted position into the extended position, the resilient arm 51 passes the edge 40a and stops at the height of the flap 46. This position is shown in FIG. 40. The auto-injector 1000 is now in the third locked state. The resilient arm 51 and optionally also the flap 46 may be biased in inward radial direction. Thus, the resilient arm 51 and the flap 46 move radially inwardly and each reach a second radial position. This is possible because the elements are no longer held by the second portion 22 of the rotating collar 2 in their respective first radial position.

The flap 46 being in the second radial position does no longer terminate flush with the edge 40a of the drive spring holder 4. Thus, when moving the needle shroud 5 from the extended position towards the retracted position, the resilient arm 51 will hit against the edge 40a which prevents a further movement of the needle shroud 5 in proximal direction P.

### 6. Drop Protection Mechanism

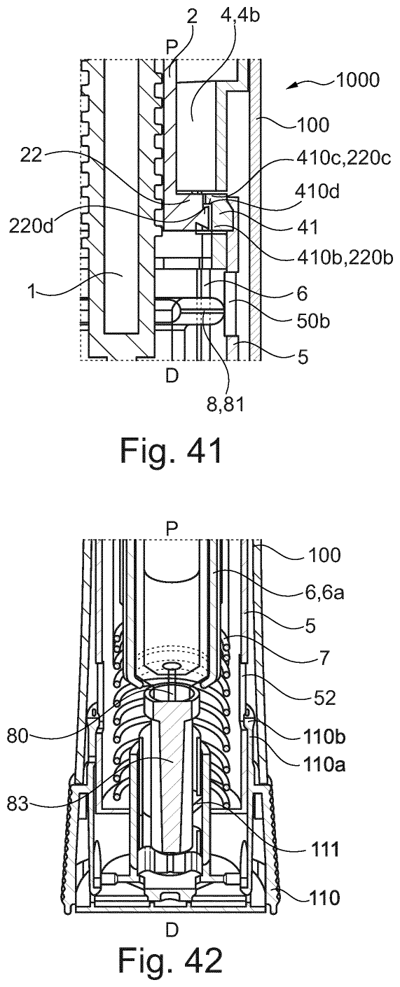

An exemplary embodiment of a drop protection mechanism is described in further detail in the following in connection with the FIGS. 41 and 42. The drop protection mechanism shall prevent the release of the first locking mechanism when the auto-injector 1000 is unintentionally dropped. In fact, when the auto-injector 1000 of the exemplary embodiments described herein is in the first locked state, a movement of the rotating collar 2 in proximal direction P would result in a release of the first locking mechanism.

FIG. 41 shows a section of the auto-injector 1000 of the first and second exemplary embodiments in cross-sectional view illustrating a first part of the drop protection mechanism. In the first locked state of the auto-injector 1000 and when the needle shroud 5 is still in the extended position (initial position), the second portion 22 and the resilient arm 41 engage with each other (protrusion 410 projects into recess 220) and this engagement is retained by the needle shroud 5 holding the resilient arm 41 in its radial position as explained in connection with the first locking mechanism. The engagement, however, also establishes an axial-lock interface preventing the rotating collar 2 from an axial movement at least in proximal direction P.

For this purpose, the protrusion 410 of the resilient arm 410 is a stepped protrusion having two sections 410b, 410c (see also FIG. 49). The recess 220 in the second portion 22 of the rotating collar 2 is a stepped recess also having two sections 220b, 220c. The sections 410b, 410c are connected by a surface 410d running essentially perpendicularly to the longitudinal axis. The sections 220b, 220c are also connected by a surface 220d running essentially perpendicularly to the longitudinal axis. The surface 220d is located more distally than the surface 410d. These surfaces 220d, 410d abut or hit against each other when the rotating collar 2 is moved in proximal direction P and, in this way, the rotating collar 2 is prevented from moving in proximal direction P as long as the protrusion 410 projects into the recess 220.

The first part of the drop protection mechanism described in connection with FIG. 41 could, however, be released when the needle shroud 5 would unintentionally be moved in proximal direction P. Therefore, in one exemplary embodiment, the drop protection mechanism comprises a second part illustrated in connection with FIG. 42.

FIG. 42 shows a section of the auto-injector in a cross-sectional view with the cutting plane running parallel to the longitudinal axis A. Shown is the distal end of the auto-injector with the cap 110 still being coupled to the housing 100. The cap 110 is in its most proximal position and cannot be moved further in proximal direction P with respect to the housing 100 since it hits against the housing 100 when moved in this direction. The cap 110 comprises a radially displaceable cap-lock element 110a, namely a resilient arm 110a, with a protrusion 110b protruding radially inwardly and engaging into a cap-lock element 52, namely a recess 52, particularly a cut-out 52, in the needle shroud 5.

In FIG. 42, the auto-injector 1000 is shown when it is dropped which results in a proximal movement of the needle shroud 5. The needle shroud 5, particularly an edge of the needle shroud 5 delimiting the recess 52 in distal direction D, hits against the protrusion 110b due to its proximal movement. This prevents a further movement of the needle shroud 5 in proximal direction P as long as the cap 110 is coupled to the housing 100. Thus, the needle shroud 5 cannot reach the retracted position in which it would no longer hold the resilient arm 41 in its radial position.

In the position shown in FIG. 42, the resilient arm 110a cannot or only slightly be moved in outward radial direction as the housing 100 circumferentially surrounds the resilient arm 110a and abuts or almost abuts against the resilient arm 110a thereby preventing an outward radial movement of the resilient arm 110a.

The protrusion 110b is located at a proximal end of the resilient arm 110a of the cap 110. Normally, when the drug delivery device is not dropped, the edge of the needle shroud 5 delimiting the recess 52 in distal direction D is located further distal as to what is shown in FIG. 42. When removing the cap 110, the cap 110 is moved in distal direction D until the protrusion 110b hits against said edge of the recess 52. The resilient arm 110a can then move in radial outward direction, because in this position of the cap 110, the housing 100 does not prevent the resilient arm 110a from being moved radially outwardly. The resilient arm 110a can disengage from the recesses 52 and the cap 110 can be completely removed. The protrusion 110b has a beveled surface (slide feature) which hits against the edge of the recess 52 and thereby forces the resilient arm 110a to deflect radially outwardly when the cap 110 is moved in distal direction D.

### 7. Subassemblies, Assembling and Second Locking Mechanism

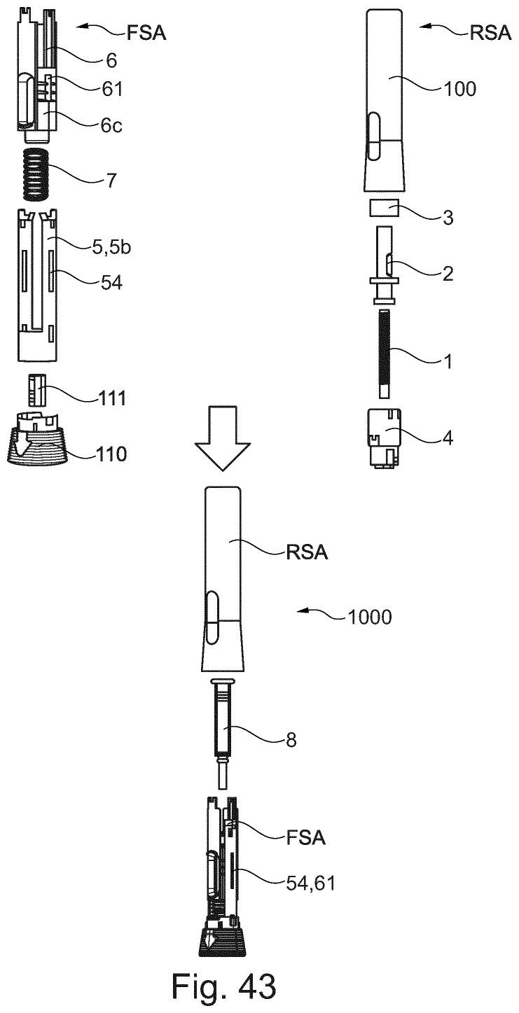

FIG. 43 shows the front subassembly FSA (also referred to as release subassembly FSA or container-holder subassembly FSA) and the rear subassembly RSA (also referred to as drive subassembly RSA) of the auto-injector according to the first exemplary embodiment in an exploded view as well as a position during assembling the front subassembly FSA and the rear subassembly RSA to an auto-injector 1000. These figures correspond to the FIGS. 13, 15 and 16. It is therefore mainly referred to the description in connection with these figures.

What can be seen in FIG. 43 is that the support portion 6c of the syringe holder 6 comprises a first rotation-lock features 61 in the form of protrusions 61 or ribs 61 which protrude in outward radial direction and which have a main extension direction along the longitudinal axis. These ribs 61 are configured to engage with second rotation-lock features 54 in the form of recesses 54, particularly slots 54, in the arms 5b of the needle shroud 5. The recesses 54 are also elongated with a main extension direction along the longitudinal axis and are longer than the ribs 61 so that, when engaged, a relative axial movement between the needle shroud 5 and the syringe holder 6 is possible.

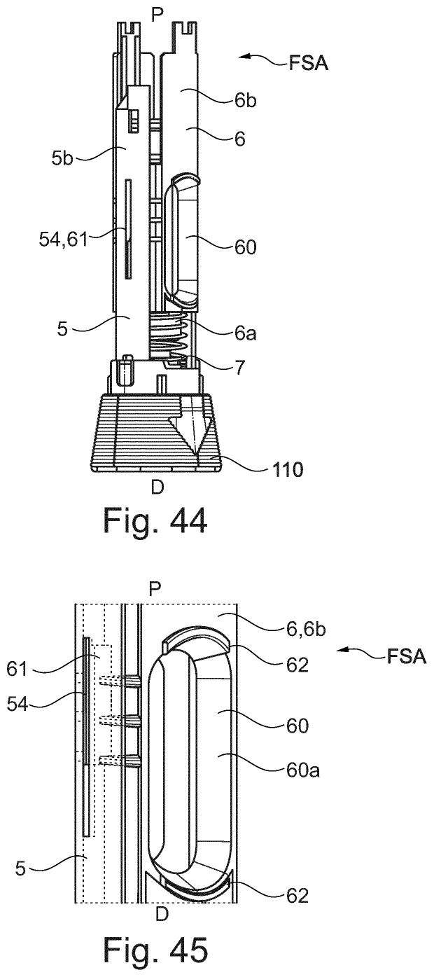

FIG. 44 shows the front subassembly FSA in perspective view. As previously described, the needle shroud 5 comprises two arms 5b which are positioned, along the angular direction, between two arms 6b of the syringe holder 6. The arms 6b of the syringe holder 6 project beyond the arms 5b of the needle shroud 5 in proximal direction P. The needle shroud 5 and the syringe holder 6 are coupled by the shroud spring 7 and the rotation-lock features 61, 54 so that the needle shroud 5 can be moved axially but not rotationally with respect to the syringe holder 6.

In FIG. 45, a section of the front subassembly FSA of FIG. 44 is shown. Windows 60 are formed in the arms 6b of the syringe holder 6, through which a syringe or medicament container located inside the syringe holder 6 can be investigated. The windows 60 are delimited by a wall portion 60a of the syringe holder 6. The diameter of the windows 60 decreases in inward radial direction.

The syringe holder 6 further comprises snap features 62, namely ribs, protruding in outward radial direction. A respective snap feature 62 is located at the distal end and at the proximal end of the window 60. The snap features 62 are configured to engage with the housing 100 to fix the syringe holder 6 to the housing 100 such that an axial and a rotational movement of the syringe holder 6 with respect to the housing 100 is prevented.

In FIG. 45, the ribs 61 project into the recesses 54 allowing an axial movement of the needle shroud 5 with respect to the syringe holder 6 but preventing a rotational movement of the needle shroud 5 with respect to the syringe holder 6. For that purpose, the width of the recesses 54 might be substantially as great as the width of the ribs 61.

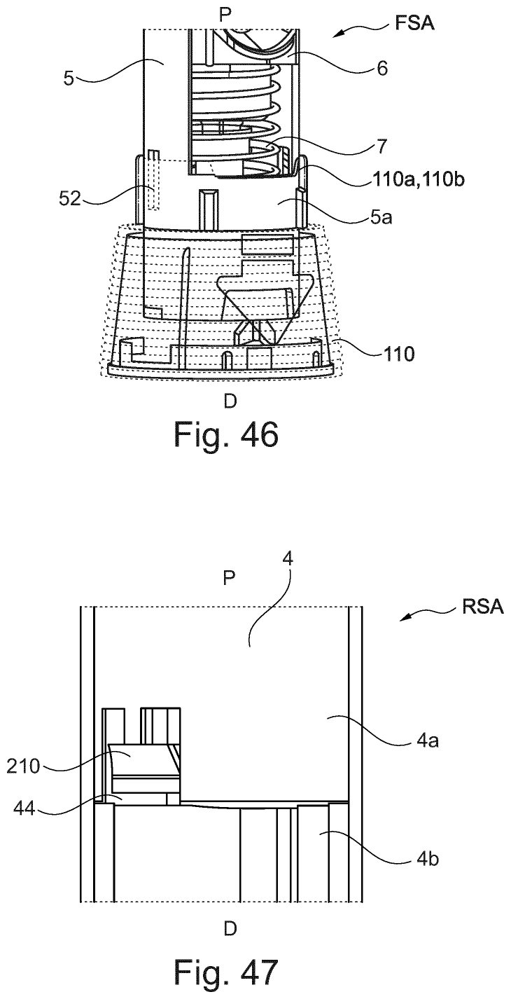

FIG. 46 shows a detailed view of the distal end of the front subassembly FSA with the cap 110 attached to the needle shroud 5. The protrusions 110b of the resilient arms 110a project into the recesses 52 of the needle shroud 5 so that the cap 110 is loosely held in position with respect to the needle shroud 5.

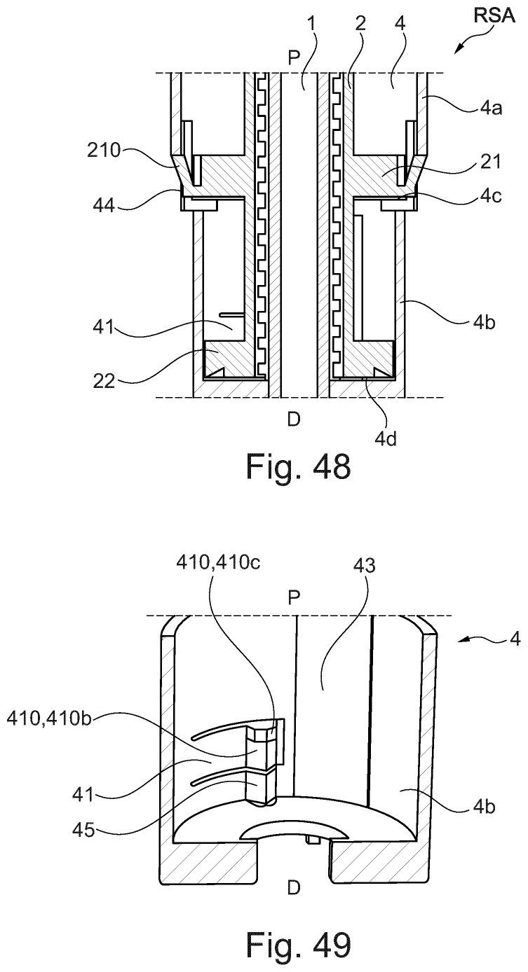

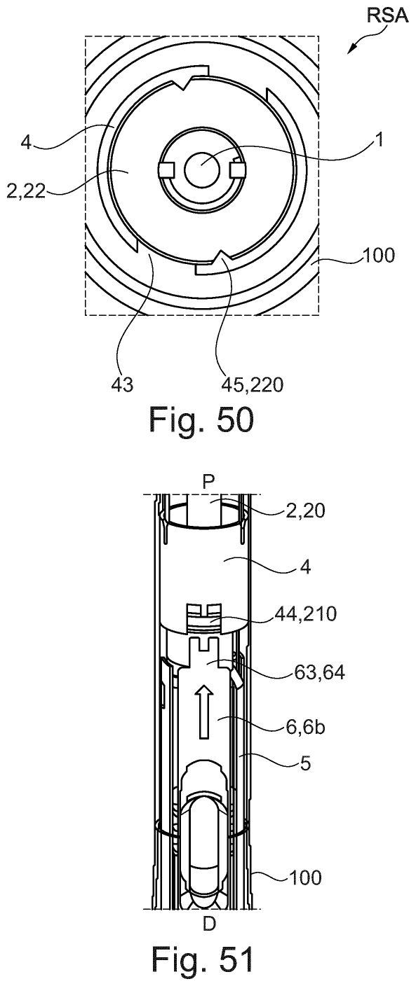

FIG. 47 shows a section of the rear subassembly RSA in perspective view. FIG. 48 shows the rear subassembly RSA in cross-sectional view with the longitudinal axis A running in the cutting plane. FIG. 50 shows the rear subassembly RSA in a cross-sectional view with the cutting plane running perpendicularly to the longitudinal axis A. An exemplary embodiment of the second locking mechanism is illustrated on the basis of these figures.

As can be seen in FIG. 47, a recess 44, particularly a cut-out, is formed in the first section 4a of the syringe holder 4. The first portion 21 of the rotating collar 2 comprises a displaceable axial-lock element 210 in form of a resilient arm 210 or clip 210. The resilient arm 210 is displaceable in radial direction. The resilient arm 210 is configured to project into the recess 44 when it is in a first radial position. In this case, the rear subassembly RSA is in a second locked state. The engagement of the resilient arm 210 and the recess 44 establishes an axial-lock interface and prevents a proximal movement of the rotating collar 2 with respect to the drive spring holder 4, because, when moving the rotating collar 2 in proximal direction P, the resilient arm 210 hits against an edge of the drive spring holder 4 delimiting the recess 44 in proximal direction P. This is one part of the second locking mechanism, also referred to as axial-locking mechanism.

As can be seen in FIG. 48, in the second locked state, the second portion 22 of the rotating collar 2 abuts against the second bottom ring 4d of the drive spring holder 4. The first portion 21 of the rotating collar 2 abuts against the first bottom ring 4c of the drive spring holder 4.

The second locking mechanism comprises also a protrusion 45 (see also FIG. 49) which is part of the second portion 4b of the drive spring holder 4 protruding radially inwardly. The protrusion 45 is not movable in any direction with respect to the rest of the drive spring holder 4. The protrusion 45 may have the same form as the first section 410b of the protrusion 410 of the resilient arm 41. The protrusion 45 is offset in distal direction D with respect to the resilient arm 41 or the protrusion 410, respectively. Furthermore, the second locking mechanism comprises the second section 22 of the rotating collar 2 with the above described recess 220 forming also part of the previously described first locking mechanism.

In the second locked state, the protrusion 45 projects into the recess 220 (see FIG. 50) thereby establishing a rotation-lock interface. This engagement prevents a rotation of the rotating collar 2 (the biased torsion drive spring 3 may already induce a torque onto the rotating collar 2 in the second locked state). This is another part of the second locking mechanism, also referred to a second rotation-locking mechanism.

The second rotation-locking mechanism does not need the needle shroud 5 for retaining the second locked state as the protrusion 45 is not displaceable in radial direction. Thus, as long as the rotating collar 2 is not moved in proximal direction P, a rotation of the rotating collar 2 is not possible.

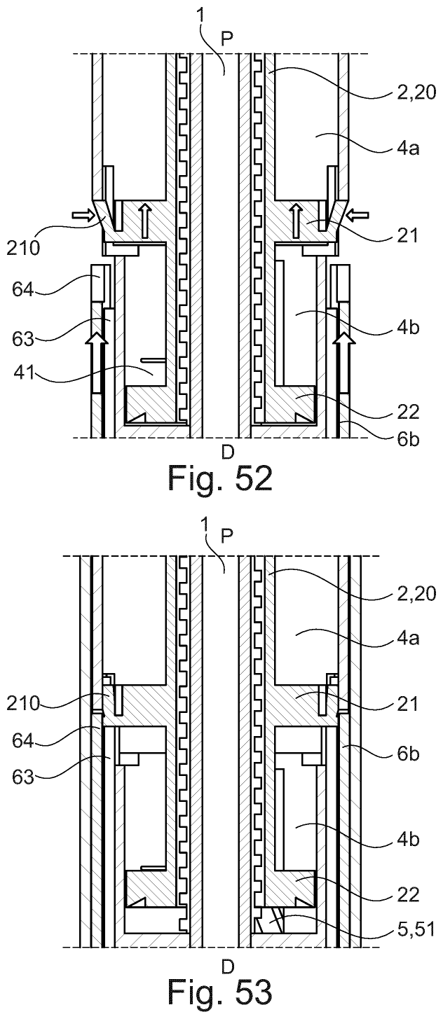

FIG. 51 shows a position in the assembly of the auto-injector, in which the rear subassembly and the front subassembly of the previous figures are telescoped into each other. FIG. 52 shows the same position in the assembling as FIG. 51 but in a cross-sectional view.

As can be seen in FIG. 52, the arms 6b of the syringe holder 6 each comprise or form at their proximal ends a push element 63 and a release element 64. The release element 64 protrudes beyond the push element 63 in proximal direction P. Moreover, the push element 63 is offset in inward radial direction with respect to the release element 64. When being telescoped into each other, the release element 64 first hits against the resilient arm 210 and forces the resilient arm 210 to move radially inwardly so that the axial-locking mechanism is released. This is realized by the resilient arm 210 having a beveled surface tilted with respect to the longitudinal axis so that a force acting on the beveled surface in proximal direction P pushes the resilient arm 210 in inward radial direction.

At the same time or later during telescoping the rear subassembly into the front subassembly, the push element 63 hits against the first section 21 of the rotating collar 2 and pushes the rotating collar 2 in proximal direction P (see also FIG. 53). This results in a release of the second rotation-locking mechanism and a transfer from the second locked state into the first locked state. The first locked state is occupied because the pushing of the rotating collar 2 in proximal direction P is accompanied with the needle shroud 5 being brought in the position where it holds the resilient arm 41 in its first radial position. Pushing the rotating collar 2 in proximal direction P during assembly has as a consequence that the recess 220 in the second portion 22 disengages with the protrusion 45 but before engages with the protrusion 410 of the resilient arm 41 (see also FIG. 49).

### 8. Feedback Mechanism

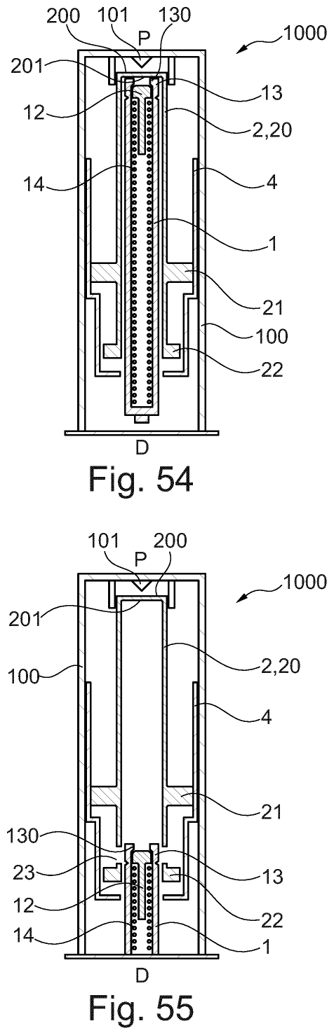

FIGS. 54 to 56 illustrate an exemplary embodiment of a feedback mechanism. Such a feedback mechanism can be used in any one of the exemplary embodiments of a drug delivery device described herein.

FIG. 54 shows a section of an exemplary embodiment of a drug delivery device/auto-injector 1000 with such a feedback mechanism. In FIG. 54, the auto-injector 1000 may be in the first locked state (initial state).

The feedback mechanism comprises a plunger rod 1 received in a rotating collar 2. The rotating collar 2 may be designed as described in connection with the previous figures. Particularly, the rotating collar 2 is a sleeve. The plunger rod 1 is hollow, e.g. hollow cylindrically-shaped. A feedback energy member 14 in the form of a spring 14, e.g. compression spring, is received in the plunger rod 1, i.e. in a cavity thereof. Furthermore, a feedback element 12 in the form of a piston 12 is received in the plunger rod 1. The spring 14 is connected to the piston 12 and to the plunger rod 1 and is compressed. The spring 14 induces a force onto the piston 12 pointing in proximal direction P, i.e. the piston 12 is biased in proximal direction P relative to the plunger rod 1.

The plunger rod 1 comprises displaceable arms 13 oriented in axial direction. The displaceable arms 13 may be resilient arms 13 and are located at the proximal end of the plunger rod 1. The displaceable arms 13 each comprise a stop feature 130 in the form of a protrusion 130 at their respective proximal end. The displaceable arms 13 together with their protrusions 130 are each displaceable in radial direction. The displaceable arms 13 are each in a first radial position. They may be biased in the outward radial direction. However, the displaceable arms 13 are held in the first radial position by a sidewall of the rotating collar 2 circumferentially surrounding the plunger rod 1 at least at the height of the displaceable arms 13.

The protrusions 130 of the displaceable arms 13 project into the cavity of the plunger rod 1. The proximal end of the piston 12 abuts against the protrusions 130. This prevents a movement of the piston 12 in proximal direction P driven by the spring 14 beyond the protrusions 130.

As visible in FIG. 54, the piston 12 and the protrusions 130 each comprise slide features in the form of beveled surfaces tilted with respect to the longitudinal axis and the radial direction. The piston 12 and the protrusions 130 abut against each other at the beveled surfaces which biases the protrusions 130 or the displaceable arms 13, respectively, in outward radial direction.

FIG. 55 shows the auto-injector 1000 in the released state. The torsion drive spring induces a torque onto the rotating collar 2 which starts rotating in a first rotational direction and thereby the plunger rod 1 is moved in distal direction D. The biased spring 14 and the piston 12 move together with the piston rod 1 in distal direction D. During the movement, the displaceable arms 13 of the plunger rod 1 are held in the first radial position by the sidewall of the rotating collar 2 still circumferentially surrounding the resilient arms 13.

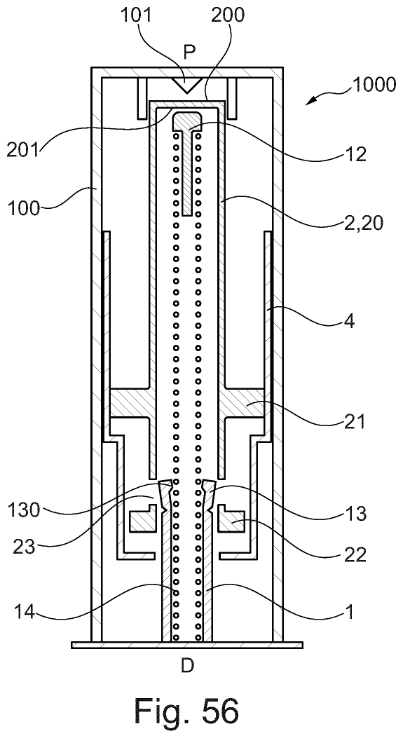

In a region of the distal end of the rotating collar 2, namely in the region between the first section 21 and the second section 22, the side wall of the rotating collar 2 is interrupted by a recess 23. When the plunger rod 1 reaches a feedback position, the displaceable arms 13 or the protrusions 130, respectively, axially and rotationally overlap with this recess 23. Thus, the displaceable arms 13 are no longer held in the first radial position. As they are biased radially outwardly, the displaceable arms 13 leave the first radial position and move in outward radial direction into a second radial position. In the second radial position, the piston 12 is no longer prevented from moving in proximal direction P relative to the plunger rod 1 driven by the spring 14 beyond the protrusions 130. This is illustrated in FIG. 56.

In FIG. 56, it can be seen that the piston 12, due to the force induced by the spring 14, moves in proximal direction P, thereby leaves the plunger rod 1 and finally hits against a proximal end 201 of the rotating collar 2 forming an impact feature 201. This hit may cause an audible and/or tactical feedback which indicates the user the end of the drug delivery process. For example, the auto-injector is designed such that the piston 12 hitting the impact feature 201 creates a noise of at least 20 dB.

### 9. Third Exemplary Embodiment of a Drug Delivery Device

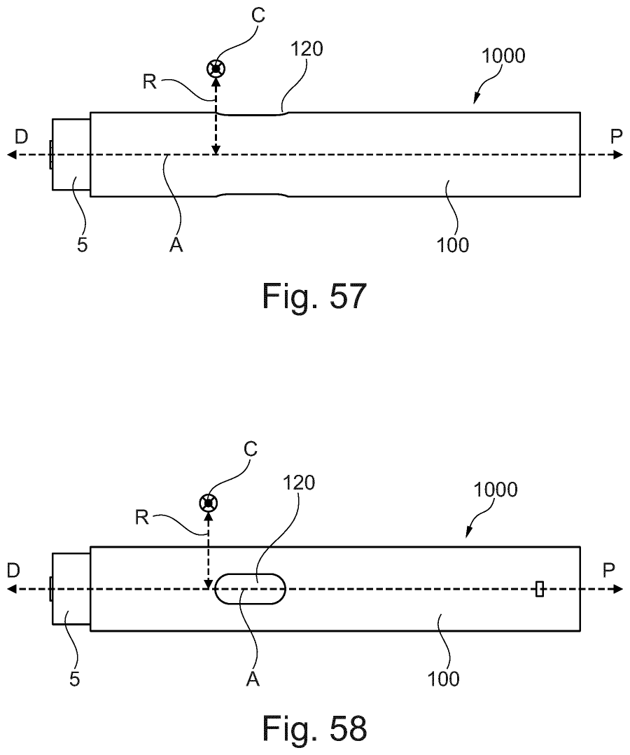

FIGS. 57 and 58 show a third exemplary embodiment of a drug delivery device 1000. FIG. 57 is a side view and FIG. 58 is a side view rotated by 90° around the longitudinal axis A with respect to FIG. 57. The drug delivery device 1000 is an auto-injector.

The auto-injector 1000 comprises a housing 100 with a window 120. The window 120 may be used for inspecting the fill level of a medicament container or a syringe or a progress of a stopper inside the housing 100 or the drug clarity or the degradation of the drug.

The auto-injector 1000 further comprises a protection member 5 in the form of a needle shroud 5 which is telescopically coupled to the housing 100 and is axially movable with respect to the housing 100.

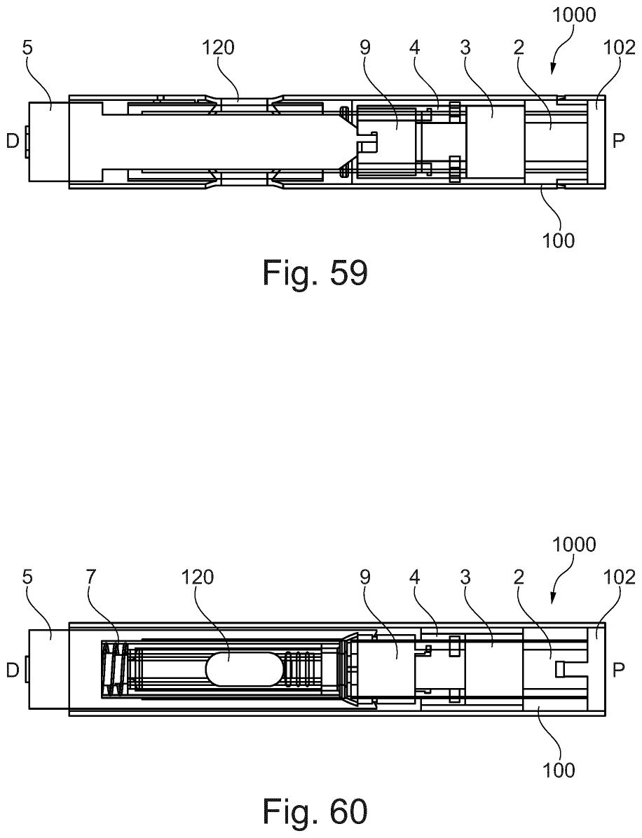

FIGS. 59 and 60 show the auto-injector 1000 of FIGS. 57 and 58 in the same views but now with the housing 100 being indicated semi-transparent which allows to see further members and elements of the auto-injector 1000. It can be seen, that the auto-injector 1000 further comprises a rear cap 102 which closes the housing 100 at the proximal end. Furthermore, the auto-injector 1000 comprises a drive spring holder 4, which is hollow, e.g. a sleeve. A torsion drive spring 3 is received in the drive spring holder 4. The torsion drive spring may be a spiral torsion spring. A rotating collar 2 is received in the torsion drive spring 3 and the drive spring holder 4. Moreover, a moveable member 9, also referred to as activation element 9, in the form of an activation collar 9 is provided. The activation collar 9 is releasably axially coupled to the needle shroud 5 so that an axial movement of the needle shroud 5 induces an axial movement of the activation collar 9. The activation collar 9 is located downstream of the torsion drive spring 3 in distal direction D and circumferentially surrounds a portion of the rotating collar 2.

Furthermore, the auto-injector 1000 comprises a shroud spring 7 which couples the needle shroud 5 to the housing 100. The coupling via the shroud spring 7 is such that a proximal movement of the needle shroud 5 with respect to the housing 100 compresses the shroud spring 7. This compression biases the needle shroud 5 in distal direction D relative to the housing 100.

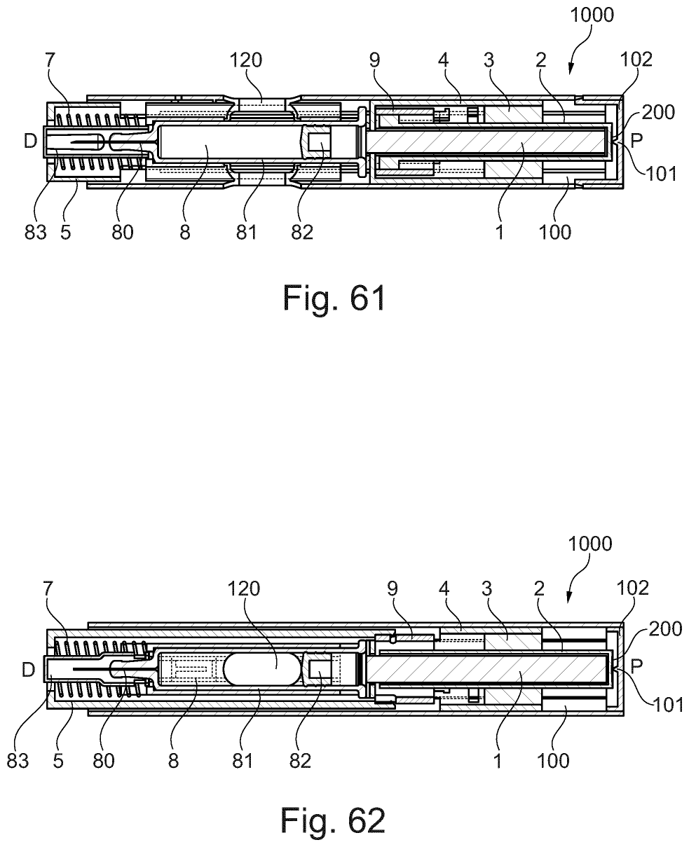

FIGS. 61 and 62 show the auto-injector 1000 of FIGS. 57 and 58 in the same views but now in a cross-sectional view with the cutting plane comprising the longitudinal axis A. In this view, it can be seen that the auto-injector 1000 further comprises a plunger rod 1. The plunger rod 1 is received in the rotating collar 2 and is circumferentially surrounded by the rotating collar 2. Only a small portion of the plunger rod 1 (less than 50% of its length) projects out of the rotating collar 2 in distal direction D. In proximal direction P, the rotating collar 2 is closed and the plunger rod 1 does not project beyond the proximal end of the rotating collar 2. The plunger rod 1 is longer, measured along the longitudinal axis, than the rotating collar 2.

The housing 100, the housing element 4, the plunger rod 1, the rotating collar 2, the needle shroud 5 and the activation element 9 may all comprise or consist of plastic. All these members may each be formed in one piece. The drive spring 3 and the shroud spring 7 may comprise or consist of a metal, e.g. steel.

It can be seen in FIGS. 61 and 62 that a medicament container 8, in the present case a syringe 8, is arranged in the housing 100. This syringe 8 may be arranged axially and/or rotationally and/or radially fixed with respect to the housing 100. The syringe 8 comprises a cartridge 81 filled with a drug, a needle 80 and a stopper 82. The needle 80 is arranged at a distal end of the syringe 8. The stopper 82 seals the cartridge 81 in proximal direction P. When moving the stopper 82 in the distal direction D, the drug stored in the cartridge 81 is pressed out of the syringe 8 through the needle 80.

In FIGS. 61 and 62 it can be further seen that the needle 80 is covered by a needle shield 83 which encapsulates the needle 80 and projects beyond the needle 80 in distal direction D. The needle shield 83 may be removed before using the auto-injector 1000.

For using the auto-injector 1000, the distal end of the auto-injector 1000 formed by the needle shroud 5 may be pressed against a body, e.g. a human body. As a consequence of that, the needle shroud 5 moves from its extended position in the proximal direction P with respect to the housing 100. This results in the needle 80 being exposed and projecting in distal direction D so that it can now pierce into the tissue of the body.

In the position shown in FIGS. 61 and 62, the auto-injector 1000 is still in an initial state, in the following referred to as locked state, in which the torsion drive spring 3 is biased and induces a torque onto the rotating collar 2. A locking mechanism, however, prevents the rotating collar 2 from a rotational movement. The locking mechanism will be explained in more detail further below.

In the locked state, a proximal end of the rotating collar 2 may be axially spaced from a proximal end-stop of the housing 100. This allows an axial movement of the rotating collar 2 in proximal direction P. Moreover, in the locked state, a distal end of the plunger rod 1 is axially spaced from the stopper 82 of the syringe 8. Thus, the plunger rod 1 can axially move in the distal direction D for a predetermined distance before hitting the stopper 82.

The needle shroud 5 may be moved in the proximal direction P into a retracted position. This releases the locking mechanism so that the rotating collar 2 is no longer prevented from rotating. The auto-injector switches from the locked state into a released state. The torque induced by the torsion drive spring 3 onto the rotating collar 2 forces the rotating collar 2 to rotate in a first rotational direction (clockwise or counterclockwise). For example, the rotating collar 2 rotates several times around its rotational axis. A drive mechanism, e.g. the drive mechanism described before, converts the rotation of the rotating collar 2 into an axial movement of the plunger rod 1 in the distal direction D. After having moved the predetermined distance in the distal direction D, the plunger rod 1 hits the stopper 82 of the syringe 8 and can now push the stopper 82 in distal direction D which results in the drug in the cartridge 81 being pressed out through the needle 80 into the tissue.

The rotating collar 2 may not only rotate but also moves in proximal direction P until the proximal end of the rotating collar 2 hits the proximal end-stop of the housing 100. The end-stop comprises is a protrusion 101 which tapers in distal direction D. The protrusion 101 may be a cone. The proximal end of the rotating collar 2 comprises an indentation 200. For example, the surface of the proximal end of the rotating collar 2 is concavely shaped. The protrusion 101 can penetrate into the indentation 200 when the proximal end of the rotating collar 2 hits the end-stop of the housing 100. The protrusion 101 and the indentation 200 may each be designed rotationally symmetric or circular symmetric with respect to the rotational axis of the rotating collar 2. In this way, a low friction interface is formed between the housing 100 and the rotating collar 2 so that a low friction rotation of the rotating collar 2 is enabled also when the proximal end of the rotating collar 2 abuts against the housing 100. Particularly, the radius at which the friction between the rotating collar 2 and the end-stop acts is approaching zero or is zero, therefore the resulting torque from the friction also tends to zero significantly reducing losses allowing a reduced spring force and/or enhance injection performance.

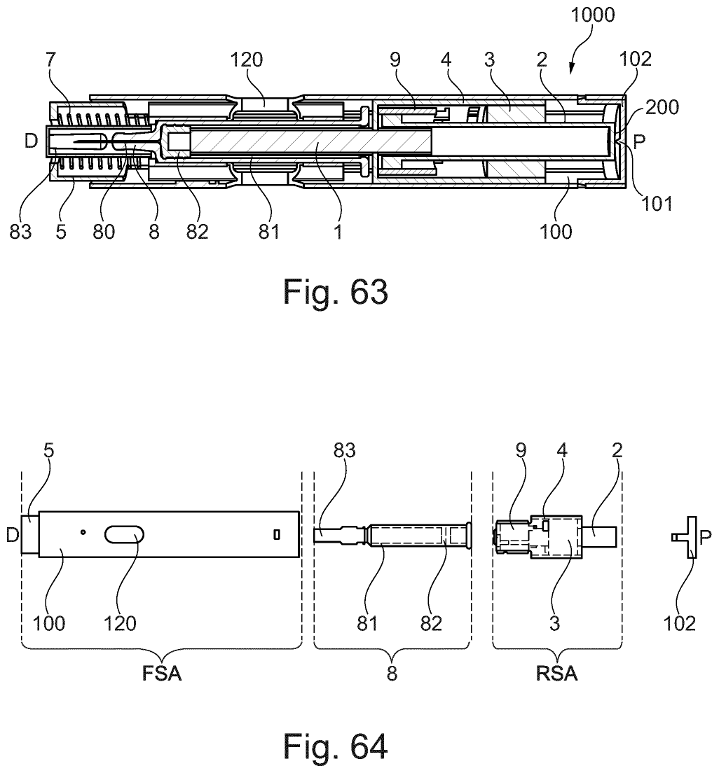

FIG. 63 shows the auto-injector 1000 according to the third exemplary embodiment in a cross-sectional view and after usage. The plunger rod 1 has hit the stopper 82 and has pushed the stopper 82 into distal direction D. As a consequence, the drug in the cartridge 82 was pushed through the needle 80 out of the syringe 8. For example, the drug was thereby injected into the tissue of the body.

FIG. 64 shows different subassemblies of the auto-injector 1000 according to the third exemplary embodiment. The auto-injector 1000 comprises a front subassembly FSA. The front subassembly FSA comprises the housing 100, the needle shroud 5 and the shroud spring 7 coupling the housing 100 and the needle shroud 5.

The auto-injector 1000 further comprises a rear subassembly RSA, with the plunger rod 1, the rotating collar 2, the torsion drive spring 3, the drive spring holder 4 and the activation collar 9.

When assembling the front subassembly FSA and the rear subassembly RSA, a syringe 8 is first telescoped into the housing 100 of the front subassembly FSA and then the rear subassembly RSA is telescoped into the housing 100. Finally the rear cap 102 is attached to the proximal end of the housing 100 and may be fixed to the housing 100 via a clip.

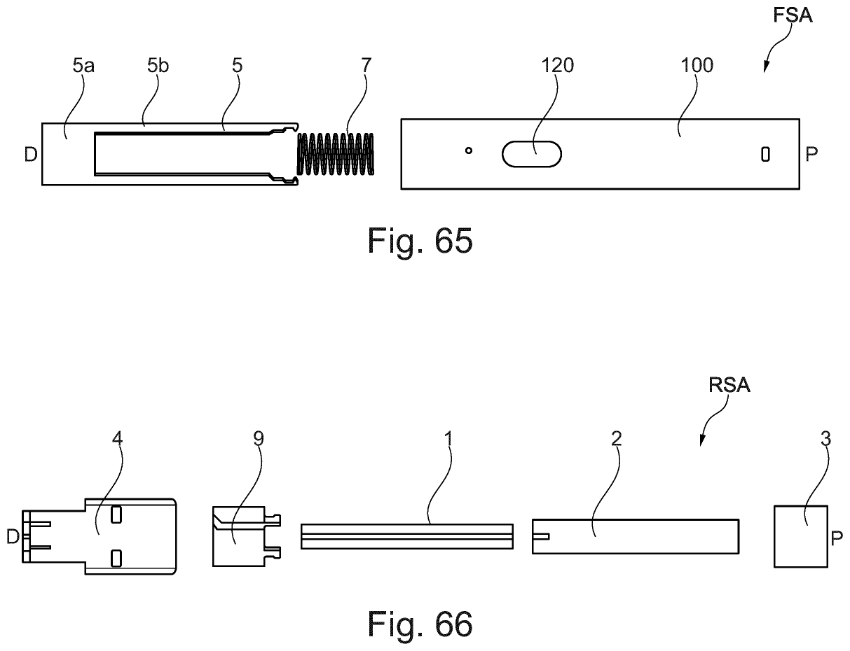

FIG. 65 shows the front subassembly FSA in an exploded view. The needle shroud 5 comprises a distal portion 5a which is shaped hollow cylindrically and into which the shroud spring 7 can be telescoped. Furthermore, the needle shroud 5 comprises two arms 5b extending from the cylindrically shaped portion 5a in proximal direction P.

FIG. 66 shows the rear subassembly RSA in an exploded view.

### 9.1 Drive Mechanism of the Drug Delivery Device According to the Third Exemplary Embodiment

The drive mechanism of the auto-injector according to the third exemplary embodiment may be designed as the previously described drive mechanism.

### 9.2 Locking Mechanism of the Drug Delivery Device According to the Third Exemplary Embodiment

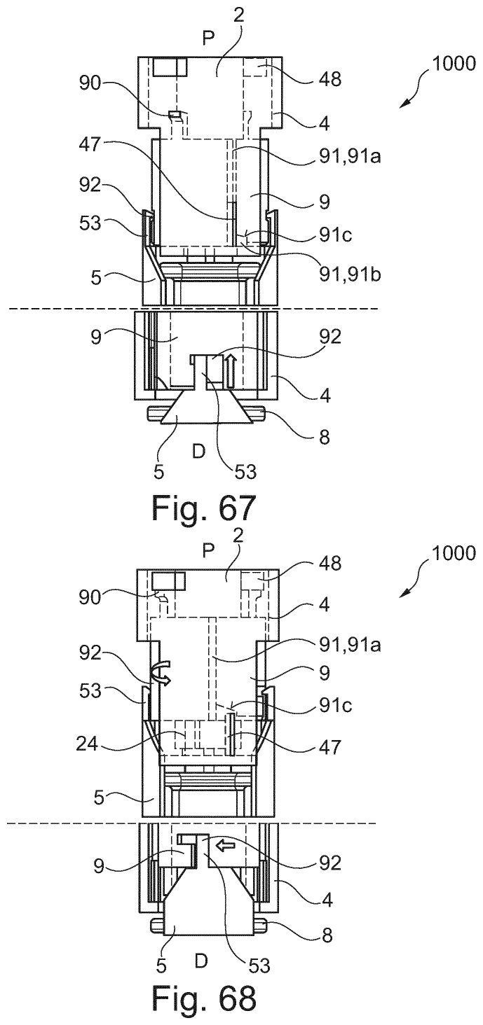

FIG. 67 shows sections of the auto-injector 1000 according to the third exemplary embodiment in the locked state.

The upper part of FIG. 67, above the horizontal dashed line, shows a section of the auto-injector 1000 in a side view. The lower part of FIG. 67, below the dashed line, shows a section of the auto-injector in a side view rotated by 90° around the longitudinal axis A with respect to the upper part.

FIG. 70 shows the auto-injector 1000, e.g. also in the locked state, in a cross-sectional view with the crossing plane being perpendicular to the longitudinal axis A.

Considering first FIG. 67, the needle shroud 5 comprises a coupling feature 53 in the form of a resilient arm 53 with a protrusion projecting radially inwardly. The activation collar 9 has a coupling feature 92 in the form of a recess 92 or opening 92. The protrusion of the resilient arm 53 projects into the recess 92. In this way, the needle shroud 5 and the activation collar 9 are axially coupled so that an axial movement of the needle shroud 5 induces an axial movement of the activation collar 9.

In the lower part of FIG. 67 it can be seen that the recess 92 is L-shaped and comprises two sections being adjacent to each other in the angular direction. In the locked state shown in FIG. 67, the resilient arm 53 engages into a first section of the recess 92. The first section of the recess 92 is bordered in proximal direction P and distal direction D by edges of the activation collar 9. Thus, an axial movement of the needle shroud 5 in proximal direction P and distal direction D results in the protrusion of the resilient arm 53 hitting either one of these edges. As a consequence, the activation collar 9 is forced to move in distal direction D when the needle shroud 5 is moved in distal direction D and the activation collar 9 is forced to move in proximal direction P when the needle shroud 5 is moved in proximal direction P. In other words, the needle shroud 5 is coupled to the activation collar 9 in proximal direction P and distal direction D.

On the other hand, the second section of the recess 92 is delimited by an edge of the activation collar 9 only in proximal direction P. In distal direction D, the second section of the recess 92 is open and not delimited by an edge of the activation collar 9. Thus, if the protrusion of the resilient arm 53 would engage into the second section of the recess 92, the protrusion would hit against an edge of the activation collar 9 when moving the needle shroud 5 in proximal direction P which would force the activation collar 9 to also move in proximal direction P. A movement of the needle shroud 5 in distal direction D, however, would result in a disengagement of the resilient arm 53 and the recess 92.

Furthermore, it can be seen in FIG. 67 that the activation collar 9 is coupled to the drive spring holder 4 via a first rotation-lock interface. The first rotation-lock interface prevents a rotation of the activation collar 9 with respect to the drive spring holder 4. On the other hand, as can be seen in FIG. 70, the rotating collar 2 and the activation collar 9 are coupled via a second rotation-lock interface. The second rotation-lock interface prevents a rotation of the rotating collar 2 with respect to the activation collar 9. Thus, in sum, a rotation of the rotating collar 2 with respect to the drive spring holder 4 is prevented by the two rotation-lock interfaces.

The first rotation-lock interface is established by a slit 91a in the activation collar 9 and a rib 47 of the drive spring holder 4 engaging into the slit 91. The rib 47 and the slit 91 are each elongated with a main extension direction along the longitudinal axis. As can be seen in FIG. 67, the slit 91a is a first section of a recess 91 in the activation collar 9. The recess 91 also comprises a second section 91b adjoining the slit 91a in distal direction D. The slit 91 has a smaller width, measured along the angular direction, than the second section 91b. The width of the second section 91b first increase in direction away from the slit 91a and then has a constant width. In this region of increasing width, the second section 91b is delimited by a beveled surface 91c of the activation collar 9 which is tilted with respect to the longitudinal axis and the rotational direction. This beveled surface 91c realizes a slide feature. In the locked state, shown in FIG. 67, the rib 47 engages into the slit 91a of the recess 91.

As can be seen in FIG. 70, the second rotation-lock interface is realized by a protrusion 93 of the activation collar 9 and a protrusion 24 of the rotating collar 2 abutting against each other in angular direction. The protrusion 93 of the activation collar 9 projects radially inwardly and the protrusion 24 of the rotating collar 2 projects radially outwardly. The protrusions 24, 93 abut against each other such that a rotation of the rotating collar 2 induced by the biased torsion drive spring 3 with respect to the activation collar 9 is prevented or blocked by the activation collar 9.

FIG. 68 shows the autoinjector 1000 in a position in which the needle shroud 5 has been moved from its extended position proximally towards the retracted position. The needle shroud 5 is now in an intermediate position between the extended position and the retracted position. In this intermediate position, the rib 47 is transferred from the slit into the second section 91b. Due to the force induced by the drive spring 3, the beveled surface 91c is pressed against the rib 47 and the rib 47 slides along the beveled surface 91c whereby the activation collar 9 rotates with respect to the drive spring holder 4 and with respect to the needle shroud 5 by a predetermined angle in the first rotational direction. This rotation happens automatically, as the torque induced by the torsion drive spring 3 is transferred via the rotating collar 2 to the activation collar 9 (via the second rotation-lock interface). After the rotation by the predetermined angle, an edge of the activation collar 9 running parallel to the longitudinal axis and delimiting the second section 91b of the recess 91 in angular direction hits against the rib 47. A further rotation of the activation collar 9 with respect to the drive spring holder 4 in the first rotational direction is then prevented.

However, the rotation of the activation collar 9 by the predetermined angle in the first rotational direction has as a consequence that the resilient arm 53 of the needle shroud 5 now engages into the second section of the recess 92 of the activation collar 9 which results in a decoupling of the activation collar 9 and the needle shroud 5 in distal direction D. In other words, the coupling of the needle shroud 5 and the activation collar 9 in distal direction D is released.

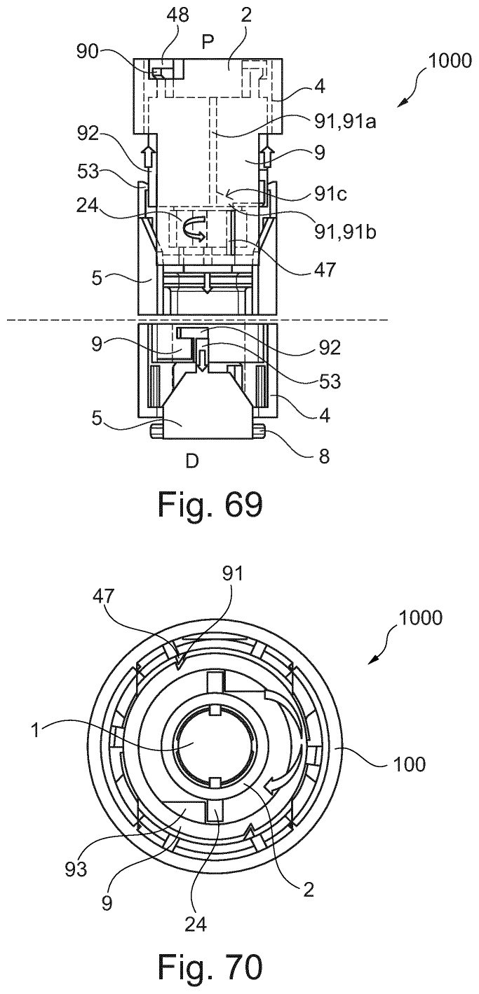

FIG. 69 shows the auto-injector 1000 in a position in which the needle shroud 5 has been further moved in proximal direction P into the retracted position which has also forced the activation collar 9 to further move in proximal direction P. In this retracted position of the needle shroud 5, the needle 80 of the auto-injector 1000 may be exposed allowing the needle 80 to pierce into a tissue of a body. In the retracted position of the needle shroud 5, the second rotation-lock interface between the activation collar 9 and the rotating collar 2 is released, i.e. the protrusion 24 and the protrusion 93 are now axially offset and do not abut against each other any longer so that the auto-injector 1000 transfers into the released state in which the rotation of the rotating collar 2 with respect to the activation collar 9 and with respect to the drive spring holder 4 is enabled. The rotating collar 2 rotates in the first rotational direction and thereby drives the plunger rod 1 in distal direction D which results in a delivery of the drug through the needle 80 (see description above).

Furthermore, the movement of the activation collar 9 further in proximal direction P had as a consequence that a second coupling feature 90 of the activation collar 9, namely a clip 90, has engaged into a coupling feature 48 of the drive spring holder 4, namely a recess 48. The engagement between the clip 90 and the recess 48 is such that a movement of the activation collar 9 in distal direction D is prevented. When moving the needle shroud 5 from the retracted position back towards or into the extended position, the activation collar 9 does not and cannot follow. A movement of the needle shroud 5 in distal direction D relative to the activation collar 9 is enabled since the resilient arm 53 engages into the second section of the recess 92 as described above.

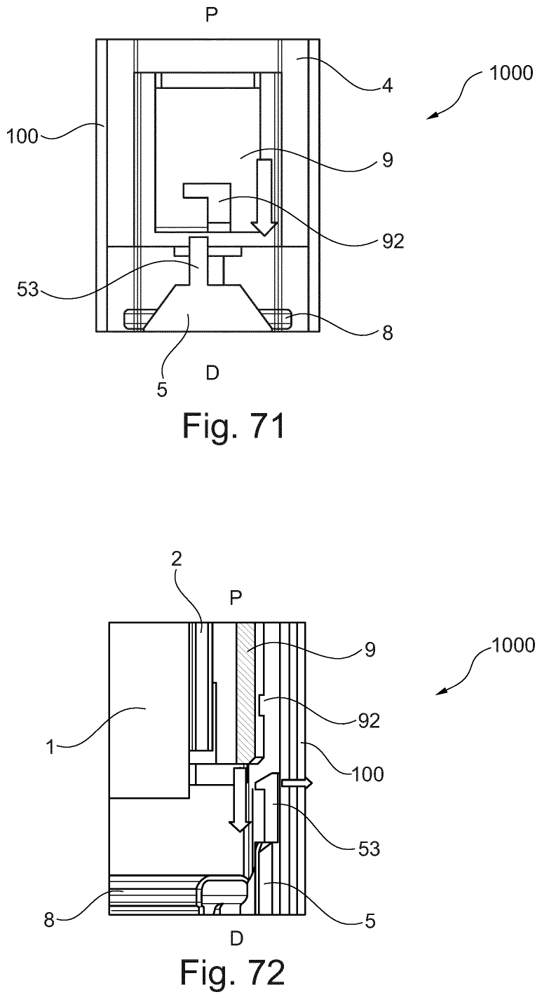

FIG. 71 to 73 show different positions during assembling the auto-injector 1000 according to the third exemplary embodiment. The rear subassembly is telescoped into the front subassembly.

FIG. 71 shows a first position, in which the needle shroud 5 of the front subassembly and the activation collar 9 of the rear subassembly are not yet coupled to each other. FIG. 71 is a side view of the auto-injector 1000 during assembling.

FIG. 72 shows the position of FIG. 71 in a cross-sectional view. It can be seen, that the resilient arm 53 of the needle shroud 5 has a slide feature in form of a beveled surface. The beveled surface is designed such that, when the beveled surface hits the distal end of the activation collar 9, a force is created pushing the resilient arm 53 in outward radial direction. The rear subassembly and the front subassembly can then further be telescoped into each other and as soon as the protrusion of the resilient arm 53 axially and rotationally overlaps with the recess 92 of the activation collar 9, it slips into this recess 92. In this way, a coupling between the activation collar 9 and needle shroud 5 is obtained.

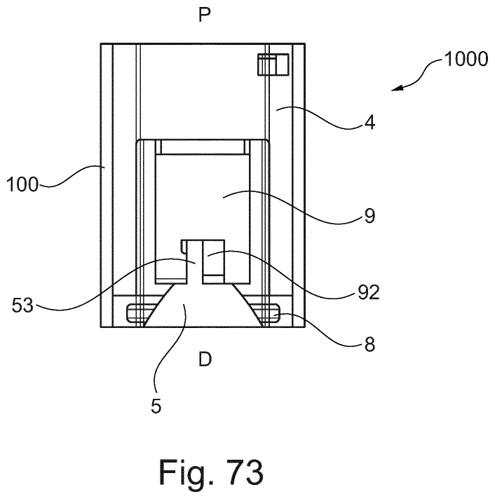

FIG. 73 shows the auto-injector after coupling of the needle shroud 5 and the activation collar 9.

### Further Explanations and Definitions

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide. Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RGO12 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful with embodiments described in the present disclosure include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

The embodiments described herein are not limited by the description in conjunction with the exemplary embodiments. Rather, any of the embodiments described herein may comprise any new feature as well as any combination of features, particularly including any combination of features in the patent claims, even if said feature or said combination per se is not explicitly stated in the patent claims or exemplary embodiments.

### REFERENCE NUMERALS

1 plunger rod
2 rotating collar
3 torsion drive spring
4 drive spring holder
4a first section of drive spring holder 4
4b second section of drive spring holder 4
4c first bottom ring of drive spring holder 4
4d second bottom ring of drive spring holder 4
5 needle shroud
5a cylindrically-shaped portion
5b arm
6 medicament container holder/syringe holder
6a cylindrically-shaped portion
6b arm
6c support portion
7 shroud spring
8 medicament container/syringe
9 activation collar
10 groove
11 external thread
12 piston
13 displaceable arm
14 feedback energy member/spring
15 shaft
21 first portion
22 second portion
22a surface
23 recess
24 protrusion
20 protrusion
40a edge in drive spring holder 4
41 resilient arm
43 recess
44 recess
45 protrusion
46 flap
47 rib
48 recess
50a wall portion
50b recess
51 resilient arm
51a ramp
52 recess
53 resilient arm
54 recess
60 window
60a wall portion
61 rib
62 snap feature
63 push element
64 release element
80 needle
81 cartridge
82 stopper
83 needle shield
90 clip
91 recess
91a slit/first section of recess 91
91b second section of recess 91
91c tilted surface
92 recess
93 protrusion
100 housing
101 protrusion
102 rear cap
110 cap
110a resilient arm
110b protrusion
111 grabber
120 window
130 protrusion
200 indentation
201 impact feature
210 resilient arm/clip
220 recess
220b first section of recess 220
220c second section of recess 220
220d surface of recess 220
221 recess
221a beveled surface
410 protrusion
410a beveled surface
410b first section of protrusion 410
410c second section of protrusion 410
410d surface of protrusion 410
411 protrusion
411a beveled surface
1000 drug delivery device/auto-injector
FSA front sub assembly
RSA rear sub assembly
α angle
D distal direction
P proximal direction
A longitudinal axis/axial direction
R radial direction
C azimuthal/rotational/angular direction

1.-18. (canceled)

19. A drug delivery device comprising:
a housing element;
a protection member arranged axially movable with respect to the housing element and configured to cover a drug delivery element; and
a movable member arranged axially and rotationally movable with respect to the housing element and arranged rotationally movable with respect to the protection member,
wherein the drug delivery device has an initial state in which (i) the protection member is in an extended position to cover the drug delivery element (ii) the protection member is movable in a proximal direction from the extended position into a retracted position to expose the drug delivery element and (iii) the protection member is coupled to the movable member in a distal direction and the proximal direction such that (a) a distal movement of the protection member in the distal direction results in a distal movement of the movable member and (b) a proximal movement of the protection member results in a proximal movement of the movable member,
wherein the drug delivery device is configured such that, starting from the initial state, a movement of the protection member in the proximal direction initiates a rotation of the movable member relative to the protection member by a predetermined angle, whereby the coupling of the protection member and the movable member in the distal direction is released such that the protection member is movable towards the extended position relative to the movable member.

20. The drug delivery device according to claim 19, comprising:
a plunger rod being arranged axially movable with respect to the housing element; and
an energy member configured to provide energy to induce an axial movement of the plunger rod in the distal direction,
wherein in the initial state, the plunger rod is coupled to the housing element via a lock interface which prevents an axial movement of the plunger rod by the energy member,
wherein the drug delivery device is configured to be switched from the initial state into a released state by moving the protection member from the extended position into the retracted position, and
wherein in the released state (i) the lock interface is released such that an axial movement of the plunger rod by the energy member is allowed, and (ii) the plunger rod is configured to move in the distal direction due to the energy provided by the energy member.

21. The drug delivery device according to claim 20, comprising:
a transfer member being arranged rotatably with respect to the housing element, the transfer member and the plunger rod being operatively coupled such that a rotation of the transfer member in a first rotational direction is converted into a movement of the plunger rod in the distal direction,
wherein in the released state, the energy member provides a torque onto the transfer member in which the transfer member rotates in the first rotational direction and forces the plunger rod to move axially in the distal direction.

22. The drug delivery device according to claim 21, wherein the drug delivery device is configured such that, in the released state, the transfer member moves axially with respect to the housing element.

23. The drug delivery device according to claim 22, wherein the transfer member moves in the proximal direction.

24. The drug delivery device according to claim 22, wherein:
the energy member is a drive spring (i) connected to the transfer member at a first connection point and (ii) connected to the housing element at a second connection point, and
during axial movement of the transfer member, the first connection point and the second connection point are axially moved with respect to each other.

25. The drug delivery device according to claim 21, wherein in the initial state, (i) the movable member and the housing element are coupled via a first rotation-lock interface to prevent a rotation of the movable member in the first rotational direction with respect to the housing element and (ii) the movable member and the transfer member are coupled via a second rotation-lock interface to prevent a rotation of the transfer member in the first rotational direction with respect to the movable member.

26. The drug delivery device according to claim 25, wherein:
the movable member has a first rotation-lock feature configured to engage with a rotation-lock feature of the housing element to prevent a rotation of the movable member in the first rotational direction with respect to the housing element,
the movable member has a second rotation-lock feature configured to engage with a rotation-lock feature of the transfer member to prevent a rotation of the transfer member in the first rotational direction with respect to the movable member, and
the drug delivery device is configured such that, starting from the initial state, a movement of the protection member in the proximal direction results in a disengagement of the first rotation-lock feature of the movable member and the rotation-lock feature of the housing element followed by a disengagement of the second rotation-lock feature of the movable member and the rotation-lock feature of the transfer member.

27. The drug delivery device according to claim 26, wherein a slide feature is arranged axially behind at least one of the first rotation-lock feature of the movable member and the rotation-lock feature of the housing element, the slide feature being arranged and configured such that, after disengagement of the first rotation-lock feature of the movable member and the rotation-lock feature of the housing element, the respective other rotation-lock feature abuts against and slides along the slide feature for a rotation of the movable member relative to the protection member by the predetermined angle.

28. The drug delivery device according to claim 27, wherein:
one of the first rotation-lock feature of the movable member or the rotation-lock feature of the housing element is a slit,
the other one of the first rotation-lock feature of the movable member or the rotation-lock feature of the housing element is a protrusion projecting in radial direction, and
the slide feature is a beveled surface tilted with respect to a longitudinal axis and/or the first rotational direction.

29. The drug delivery device according to claim 19, wherein the drug delivery device is configured such that starting from the initial state, a movement of the protection member with the movable member coupled thereto from the extended position towards the retracted position results in an axial coupling between the movable member and the housing element.

30. The drug delivery device according to claim 19, wherein:
the protection member comprises a coupling feature,
the movable member comprises a first coupling feature, and
the coupling feature of the protection member and the first coupling feature of the movable member are configured to releasably engage to provide the releasable coupling between the movable member and the protection member.

31. The drug delivery device according to claim 30, wherein the first coupling feature is arranged in a distal region of the movable member.

32. The drug delivery device according to claim 30, wherein:
one of the coupling feature of the protection member or the first coupling feature of the movable member comprises a first section and a second section arranged rotationally offset relative to each other,
the first and second sections are formed differently compared to each other,
when the first section is engaged with the other of the at least one coupling feature of the protection member or the first coupling feature of the movable member, the protection member and the movable member are coupled in the distal direction and the proximal direction,
when the second section is engaged with the other of the at least one coupling feature of the protection member or the first coupling feature of the movable member, the protection member and the movable member are coupled in the proximal direction and not coupled in the distal direction, and
the rotation of the movable member by the predetermined angle in the first rotational direction with respect to the protection member results in a transition of the engagement of the coupling feature of the protection member or the first coupling feature of the movable member from the first section into the second section.

33. The drug delivery device according to claim 19, wherein:
the movable member comprises a coupling feature,
the housing element comprises a coupling feature,
the coupling feature of the housing element and the coupling feature of the movable member are configured to engage each other to provide a coupling between the movable member and the housing element to prevent a movement of the movable member in the distal direction,
the drug delivery device is configured such that the coupling feature of the housing element and the coupling feature of the movable member engage each other when, starting from the initial state, the protection member with the movable member coupled thereto is moved in the proximal direction.

34. The drug delivery device according to claim 19, comprising:
a housing with the housing element fixed to or integrated in the housing; and
a medicament container with a needle,
wherein the protection member is telescopically coupled to the housing and axially movable with respect to the housing between the extended position in which the needle is covered by the protection member and the retracted position in which the needle is exposed.

35. The drug delivery device according to claim 19, comprising:
a housing with the housing element fixed to or integrated in the housing; and
a medicament container axially fixed to the housing.

36. The drug delivery device according to claim 35, wherein the medicament container comprises a needle.

37. The drug delivery device according to claim 35, wherein the medicament container comprises a medicament or a drug.

38. The drug delivery device according to claim 19, wherein the movable member is configured to be rotated during movement of the protection member in the proximal direction.
